(19) United States
(12) Patent Application Publication
(10) Pub. No.: US 2007/0117195 A1
(43) Pub. Date: May 24, 2007

(54) Integrated thermochemical and biocatalytic energy production system

(76) Inventors: Jerry Warner, McLean, VA (US); Michael R. Ladisch, West Lafayette, IN (US); Nathan S. Mosier, West Lafayette, IN (US)

Correspondence Address: BOSE MCKINNEY & EVANS, LLP, 2700 FIRST INDIANA PLAZA, 135 NORTH PENNSYLVANIA STREET, INDIANAPOLIS, IN, 46204, US

(21) Appl. No.: 11/480,808

(22) Filed: Jul. 3, 2006

Related U.S. Application Data
(60) Provisional application No. 60/696,161, filed on Jul. 1, 2005.
(60) Provisional application No. 60/696,149, filed on Jul. 1, 2005.

(51) Int. Cl. C12P 7/06; C12P 5/02; C12M 1/00

(52) U.S. Cl. 435/161; 435/289.1; 435/167

(57) ABSTRACT

A method and apparatus for treating organic wastes is provided. Organic wastes are separated into high and low moisture content organic waste streams. The low moisture content organic waste stream is subjected to a gasification process and generates a producer gas. The high moisture content organic waste is subjected to a fermentation process and produces a mixture of ethanol and water. Waste heat from the gasification process is subjected to a distillation column. Vapors recovered from the distillation column are mixed in a hydrous vapor form with the producer gas and produce fuel that can be used as an energy source.

### RELATED APPLICATIONS

This application claims priority to U.S. Provisional Patent Application Ser. Nos. 60/696,161 and 60/696,149, both filed Jul. 1, 2005, the disclosures of which are expressly incorporated herein by reference.

This invention was made with government support under grant reference number STTRA04-7019 awarded by the U.S. Army to Defense Life Sciences. The Government has or may have certain rights in the invention.

### TECHNICAL FIELD

The present invention is directed toward processing of organic waste, and more particularly to thermochemical and biocatalytic processing of organic waste to produce and capture energy.

### BACKGROUND OF THE INVENTION

Prolonged expeditionary deployments give rise to many logistical issues, including fuel supply and waste disposal. For example, military operations, such as recent operations into Southwest Asia, have required delivery of supplies, food, fuel, equipment and materials into many disperse geographic areas. One consequence has been the generation of remote waste stockpiles, including large quantities of organics. Removal of these waste stockpiles inflicts further costly and complex logistical overhead on the U.S. forces. Another significant problem with the dispersion of forces is providing access to adequate energy. Again, referring to the recent military operations in Southwest Asia, notwithstanding advance logistic and host nation resources, access to fuel, particularly during the early months of a crisis, can be difficult. Unnecessary consumption of fuel is best avoided. In addition, even a temporary loss of access to energy during military operations can be disastrous. To date, although organic wastes generated in military operations present a potential energy source, the wastes have not been effectively utilized. To the contrary, conventional waste handling has required collection and transportation of the waste from remote locations. In addition to the logistical difficulties and risks associated with removing waste from field locations, valuable energy must be consumed in the removal effort. Thus, there is a great need for a method and apparatus for disposing organic wastes while capturing the energy content of organic wastes for conversion into fuel and/or other beneficial uses.

The present invention is intended to address one or more of the problems discussed above.

### SUMMARY OF THE INVENTION

The present teachings are directed to an integrated thermochemical and biocatalytic energy production apparatus and method for extracting the energy potential of organic wastes for beneficial uses. The attached claims recite at least some of the novel aspects of the present teachings. Other novel aspects may be apparent from the description which follows and the materials appended hereto.

The integrated thermochemical and biocatalytic energy production methods and apparatuses described herein could be used to support expeditionary operations, for example military operations, to convert waste to electric power, hot water and useable fuel while minimizing costly waste removal expenses. The methods and apparatuses of the present teachings provide potential for significant cost savings in the operation and maintenance of expeditionary forces, reduce dependence and consumption of petroleum-based energy, ease transportation demands and risks associated therewith and further provide for environmentally responsible disposal of organic waste. The methods and apparatuses could also be used at fixed locations for treating and recovering energy from organic waste.

One aspect of the present invention is to combine biological and thermal processing systems to transform waste materials into high energy gas. One goal is to separate biological materials from other wastes and obtain separate streams of solids that are either gasified to form producer gas, or fermented to transform the biological material into ethanol fermentation broth. The broth is then processed into a hydrous ethanol vapor stream via distillation and mixed with the producer gas to form a high btu content gas. The gas that is formed can then drive an internal combustion engine and generator set specifically selected for this purpose. This enhanced producer gas is made from two major components, one from thermal processing of waste material that is not readily fermentable, and the other from ethanol derived by fermenting the food waste using yeast, as well as the yeast itself.

This approach avoids the need for carrying out high temperature pretreatment of cellulose-based waste materials since the cellulose portion is gasified, and the starch and carbohydrate portions are directly converted. Starch can be directly converted without prior cooking since significant preprocessing of the ingredients that make up the food (i.e., starch and sugar) makes these components readily susceptible to enzyme hydrolysis and fermentation. In comparison, cellulose is a recalcitrant material that, like starch, is a polymer of glucose. But unlike starch, cellulose has a physical crystalline structure that makes it resistant to hydrolysis. This resistance is removed if pretreatment is carried out. The current teachings avoid pretreatment and its added energy cost because the cellulose and other non fermentable components are used to directly form a gas that has combustible constituents. This obviates the need to first convert the cellulose to glucose and the glucose to ethanol.

As stated above, the present teachings are directed to the combination of biological and thermal processing so that the waste materials are transformed into a high energy gas. One approach is to use a sequential processing scheme (i.e., the inline system) where the fermentation first converts the starches and other carbohydrates in the waste into ethanol, and then gasifies the remaining solid materials that may consist of cardboard, plastic, and oils from cooking or found in the food. The waste mixture, once stripped of fermentable components, results in solids that are pelletized and used for gasification.

An alternate approach is the separation of waste food and kitchen waste from the cardboard and plastic, so that these waste streams are processed separately and in-parallel to a high energy gas. This is referred to as the bicameral system. Separation of the waste components occurs before any thermal or biological processing is carried out. This is followed by processing of cellulosic solids and plastics into pellets and then into producer gas. The transformation of kitchen and food wastes is carried out separately in a bioreactor to form ethanol.

In both the in-line and bicameral approaches, the ethanol vapors are recovered from a distillation column. These vapors are mixed, in a hydrous vapor form, with the producer gas. The resulting combined gases form the fuel for the engine, which in turn powers the generator set. In other words, the waste heat generated by the engine and the gasifier is used to make a workable process. Thus, for example, the use of energy from the gasifier/generator set to dry the wet cellulose before it enters the gasification chamber makes use of waste energy from thermal processing.

Other advantages may well be apparent to one of skill in the art upon consideration of the description of the invention and claims contained herein.

### BRIEF DESCRIPTION OF THE DRAWINGS

The above-mentioned aspects of the present teachings and the manner of obtaining them will become more apparent and the teachings will be better understood by reference to the following description of the embodiments taken in conjunction with the accompanying drawings, wherein:

FIG. 1 is a block diagram of two embodiments of an integrated thermochemical and biocatalytic energy production system in accordance with the present invention, namely a bicameral system and an in-line system;

FIG. 2 is a high level functional block diagram of an exemplary bicameral integrated thermochemical and biocatalytic energy production system in accordance with the present invention;

FIG. 3 is a more detailed functional block diagram of an exemplary bicameral integrated thermochemical and biocatalytic energy production system in accordance with the present invention;

FIGS. 4a-4c depict exemplary trailer mounted bicameral integrated thermochemical and biocatalytic energy production system apparatuses in accordance with the present invention;

FIG. 5 is a high level functional block diagram of an exemplary in-line integrated thermochemical and biocatalytic energy production system in accordance with the present invention;

FIG. 6 is a more detailed functional block diagram of an exemplary in-line integrated thermochemical and biocatalytic energy production system in accordance with the present invention;

FIG. 7 schematically represents an exemplary apparatus implementing the functional block diagram of FIG. 6;

FIG. 8 is a functional block diagram of an exemplary in-line integrated thermochemical and biocatalytic energy production system in accordance with the present invention;

FIGS. 9 and 10 are a mathematical model supporting the exemplary in-line integrated thermochemical and biocatalytic energy production system of FIG. 8;

FIG. 11 is an exemplary bicameral integrated thermochemical and biocatalytic energy production system in accordance with the present invention;

FIG. 12 is a mathematical model supporting the exemplary bicameral integrated thermochemical and biocatalytic energy production system of FIG. 11;

FIG. 13 is an exemplary model for predicting optimal ethanol production with minimal resources and energy in accordance with the present teachings;

FIG. 14 shows inputs and outputs of various components for use with the exemplary model of FIG. 13;

FIG. 15 is a high level functional and mathematical diagram of an exemplary in-line integrated thermochemical and biocatalytic energy production system in accordance with the present invention;

FIG. 16 is another high level functional and mathematical diagram of an exemplary in-line integrated thermochemical and biocatalytic energy production system in accordance with the present invention;

FIG. 17 is a high level functional and mathematical diagram of an exemplary integrated thermochemical gasification energy production system in accordance with the present teachings;

FIG. 18 is an exemplary biorefinery apparatus for performing an integrated thermochemical hybrid production system in accordance with the present teachings;

FIG. 19 shows the fermentation time course for a bench-scale (135 mL) and pilot scale (36 L) run in accordance with Example 6 of the present teachings; and

FIG. 20 shows the weights of plastic, paper, food, slop food (MRE) in a simulated waste stream in accordance with Example 6 of the present teachings.

Corresponding reference characters indicate corresponding parts throughout the several views.

### DETAILED DESCRIPTION

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

One foreseeable application of the present invention is in the processing of military field waste or input biomass for conversion into electricity. Military field waste typically consists of a combination of wet and dry organic waste. Examples of dry organic waste are fiberboard, paper, plastic (either petroleum based or bioplastics) and wood. Examples of high moisture content or wet wastes include food waste (starches, oils greases, etc.), slop food, raw agricultural products and biosolids. High moisture content waste is most efficiently processed and converted into energy using biocatalytic techniques described in greater detail below. Dry organic wastes are more effectively converted into energy using thermochemical techniques, which are described in greater detail below. Thus, optimal treatment of field wastes would include both thermochemical treatment for dry solids and biocatalytic processes for solubilized organics.

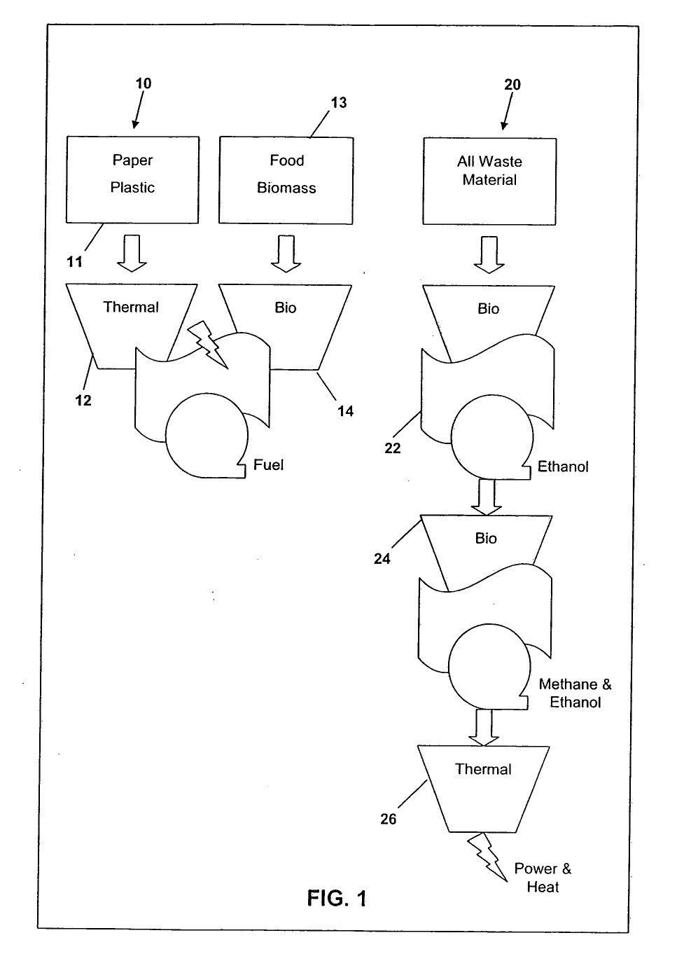

FIG. 1 illustrates two exemplary alternatives for providing both thermochemical and biocatalytic processes for the treatment of waste in accordance with the present teachings. In an exemplary “bicameral” system 10, dry wastes 11 and wet wastes 13 are separated and the dry wastes 11 are subject to thermochemical treatment 12. The wet, solubilizable wastes 13 are subjected to biocatalytic processing 14. The thermochemical processing 12 may be a pyrolosis process for the production of what is commonly known as “bio-oil.” Alternatively, the thermochemical process 12 may be a gasification process for the production of methane gas. The catalytic process may be, for example, fermentation to produce ethanol in the presence of microrganisms, enzymes or other such catalysts.

Pyrolosis is the heating of a biomass in the absence of oxygen. The lower the moisture content, the more efficient the pyrolosis process and the less energy that is required for pyrolosis to occur. Pyrolosis will produce char, permanent gasses and vapors. At ambient temperatures the vapors will condense to form a dark brown liquid commonly known as “bio-oil.” The distribution of product between liquid, char and gas on a weight basis for “slow” pyrolosis may be approximately 30%, 35% and 35%, respectively, while for “fast” pyrolosis the results may be about 75%, 12% and 13%, respectively.

Thermal chemical gasification of biomass results in the production of methane gas. Methane is produced by anaerobic digestion and other gaseous byproducts including hydrogen and carbon monoxide. Examples of gasifiers are the “BioMax Line” of Community Power Corporation of Littleton, Colo. Specific BioMax models useful according to the present teachings include the BioMax 5®, the BioMax 15® and the BioMax 50®.

The in-line system 20 illustrated in FIG. 1 provides a bioreactor 22 which initially processes the waste for the production of ethanol with an optional second bioreactor 24 for the production of methane and ethanol followed by a thermochemical reactor 26 for the production of either methane gas or bio-oil. The ethanol, bio-oil and/or methane are available for further processing, if necessary, for combustion in an electrical generator or use in other fuel powered equipment.

Ideally, for both the bicameral system 10 and the in-line system 20 waste heat generated by the thermochemical process can be utilized in the biocatalytic process and electrical energy generated by liquid and gas fuels from thermochemical and biocatalytic processes can be used to sustain the thermochemical and biocatalytic processes without the need of ongoing external energy supply. In addition, excess fuel and energy can be used to offset fuel and energy demands of the field operation.

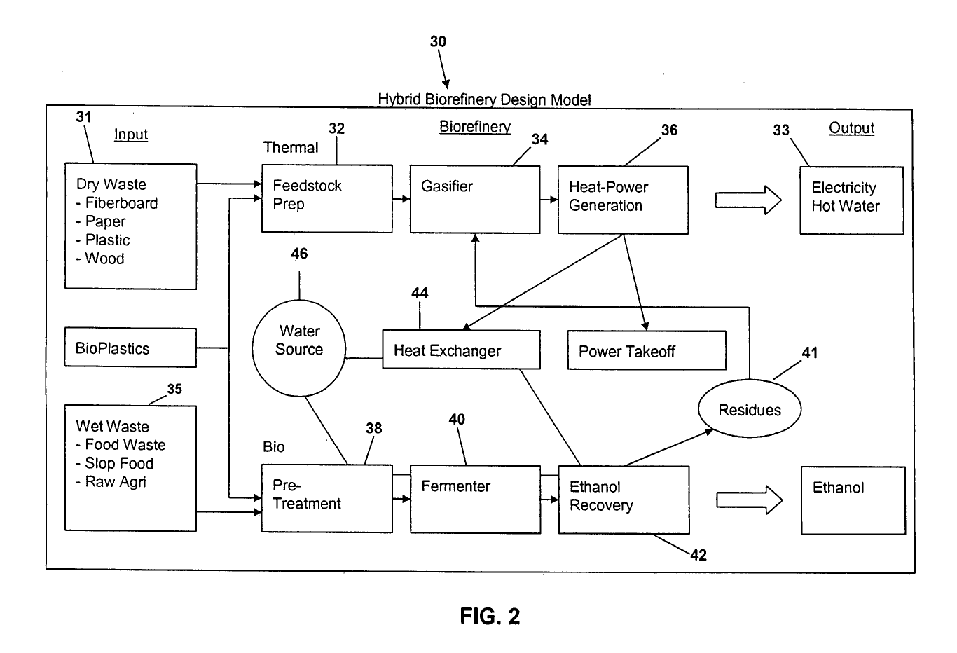

FIG. 2 illustrates a high level functional block diagram of one embodiment of a bicameral system 30 in accordance with the present teachings. In the bicameral system 30 dry high cellulose content waste 31 (e.g., materials containing cellulose, hemicelluloses and lignin—such as fiberboard, paper, plastic and wood), is introduced to a feed stock preparation station 32, which may include a grinder to break the dry waste into adequate size for treatment in the subsequent gasifier unit 34 and/or a dryer to remove excess moisture content. From the feedstock preparation station 32, the dry waste 31 is fed to the gasifier 34 where it is subject to thermochemical treatment for the production of methane gas. The methane gas is conveyed to a generator 36 for the production of electricity 33. Waste heat from the thermochemical reactions of the gasifier 34 or the generator 36 may be used for the production of hot water or in the biocatalytic process.

The biocatalytic process begins with a pretreatment station 38 for dissolving or hydrating the biomass wastes so that enzymes can more easily hydrolyze the wastes. The pretreatment station 38 may include a grinder for breaking or pulverizing solubilizable material into smaller particles for improved solubilization and a hydrolysis reaction chamber for thermal or enzyme hydrolyzation of the wet waste 35. The wet waste 35 is then delivered to a fermenter 40, where in the presence of yeast the solubilized organics are fermented into ethanol and water. Where appropriate, enzymes, such as amylases, may be provided to the fermenter for simultaneous hydrolyzation and fermentation of the wet organic waste. The addition of enzymes assist in converting starch or cellulose into monosaccharides (glucose or pentose). Such an embodiment may eliminate the need for hydrolyzation in the pre-treatment step 38. Following fermentation, the recovered mixture of ethanol and water is separated by, for example, distillation in the separator or distillation column 42. Residual solids 41 are recovered from the distillation column 42 and may be provided to the gasifier 34.

In certain embodiments, the steps of adding enzymes and fermenting the wastes may be combined so that monosaccharides are transformed to ethanol at about the same rate that they are formed. This is done to reduce the inhibition of the enzyme caused by the product that is formed by the action of the enzyme. Since the inhibition caused of yeast caused by ethanol is about 10 times lower than the inhibition caused by mon- or disaccharides, this can be advantageous since the volume of the bioreactor would be smaller. These transformations will result in about 5 to 10% by volume ethanol so that each gallon of ethanol produced would require about 9 to 18 gallons of water.

The embodiment of the bicameral system 30 illustrated in FIG. 2 includes a heat exchanger 44 for capturing waste heat from the gasifier 34 and generator 36 and conveying it to the biocatalytic process. As illustrated in FIG. 2, this can be accomplished by heating water from a water source 46 which is then used in the pre-treatment process 38. The heat exchanger 44 may also provide heat to the distillation column 42 to assist in the separation of the alcohol and water. The heat exchanger may also simply provide hot water for a wide variety of uses within the field operation.

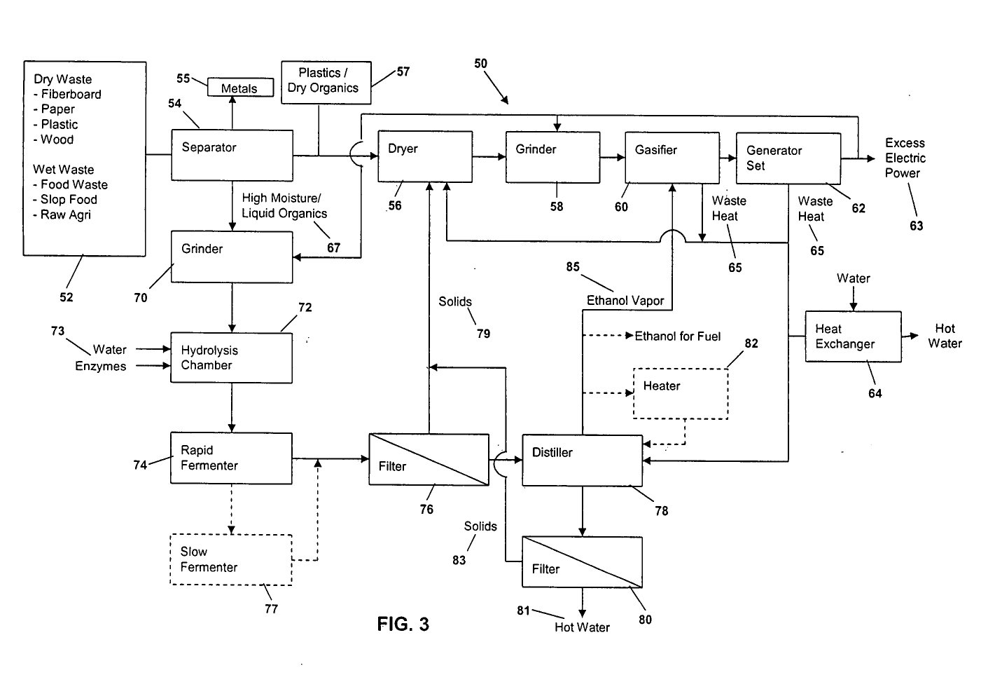

FIG. 3 illustrates in greater detail an embodiment of a bicameral integrated and biocatalytic production system in accordance with the present teachings (referred to herein as the “V2 Bicameral System”). The V2 Bicameral System receives combined dry and wet organic waste from a waste supply 52 in a separator 54. In the separator 54 metals 55 are removed from the system and plastic/dry organics 57 are separated from high moisture/liquid organics. The separator 54 may be mechanized or may be manual. The plastic/dry organics 57 are fed from the separator 54 to a dryer 56. There, excess moisture is removed and dried material is then conveyed to the grinder 58. The grinder 58 grinds the dry organics to a maximum size suitable for further processing in the gasifier 60. In the gasifier 60, methane is produced by anaerobic digestion. One suitable gasifier is the BioMax 15 of Community Power Corporation of Littleton, Colo. Methane gas produced by the gasifier 60 is conveyed to a generator set 62 for the production of electric power 63. The generator set 62 can be operated by spark-ignited engine/genset or a standard diesel powered engine/genset. Waste heat 65 from the gasifier 60 and the generator set 62 is conveyed to a heat exchanger 64 in thermal communication with the gasifier 60 and the generator set 62. Fluids such as water or air are provided to the heat exchanger 64 for heat transfer. For example, for the production of hot water or heated air.

High moisture content and liquid organics from the separator 54 are conveyed to the grinder 70 where solids are ground to a maximum size. The output of the grinder 70 is provided to a hydrolysis chamber 72 where water and enzymes 73 are provided to the high moisture/liquid organic waste to promote hydrolysis. Alternatively, thermal hydrolysis may be used. Following hydrolyzation, the waste flows to a rapid fermenter 74 where fermentation of ethanol is promoted in the presence of yeast. A mixture of ethanol and water is delivered to filter 76. Alternatively, or in addition, residual materials from the rapid fermenter 74 may be provided to the slow fermenter 77 for additional fermentation. The resulting mixed alcohol and water is conveyed to the filter 76. In the filter 76 any residual solids 79 are separated and these solids are then conveyed to the dryer 56 for thermochemical processing as described above. The separated ethanol and water is delivered to a separator in the form of a distiller 78 which yields heated water and ethanol vapor. The heated water and any remaining solids are delivered to filter 80. Hot water 81 from the filter 80 is then available for any of a variety of uses in the field camp. Solids 83 captured at the filter 80 are delivered to the dryer 56 for further processing. The ethanol vapor 85 is captured from the distiller 78 and may be further processed and utilized for fuel in the field camp. Alternatively, or in addition, the ethanol vapor 85 may be used directly, or following further processing, in the heater 82 to provide energy for the distiller 78.

The V2 Bicameral System provides for exchange of energy and fuel between the thermochemical and biocatalytic processes for enhanced efficiency and energy savings. For example, the ethanol vapor 85 from the distiller 78 may be fed directly to the gasifier 60 for the production of energy. Waste heat 65 from the gasifier 60 and the generator set 62 is captured and provided to the dryer 56 and the distiller 78. Electricity 63 generated by the generator set 62 is used to power the grinder 58 and the grinder 70. Although not illustrated, the electricity from the generator set 62 may used in a mechanized separator or to provide any other electrical energy requirements of the V2 Bicameral System.

One contemplated deployment of the V2 Bicameral System is in association with a Force Provider Module used by Army field forces. Modeling of the V2 Bicameral System indicates it is capable of converting approximately 3000 pounds of daily mixed waste into 15 kilowatts of electricity, 33 gallons of ethanol, as well as heated water to support field sanitation, showers or laundry operations. Econometric analysis predicts a cost savings of approximately $3,900 for each day the V2 Bicameral System operates. These savings are the product of reduced logistics costs for delivering fuel and the disposal of waste. The V2 Bicameral System may be deployed on an XM 1048 5-ton trailer and could be fielded as a modification upgrade for current Army trailers supporting the Force Provider Module with generators. Nominal training of existing personal would be required, but no additional man power is believed to be necessary to operate the system. The V2 Bicameral System is expected to meet all necessary environmental and safety regulations.

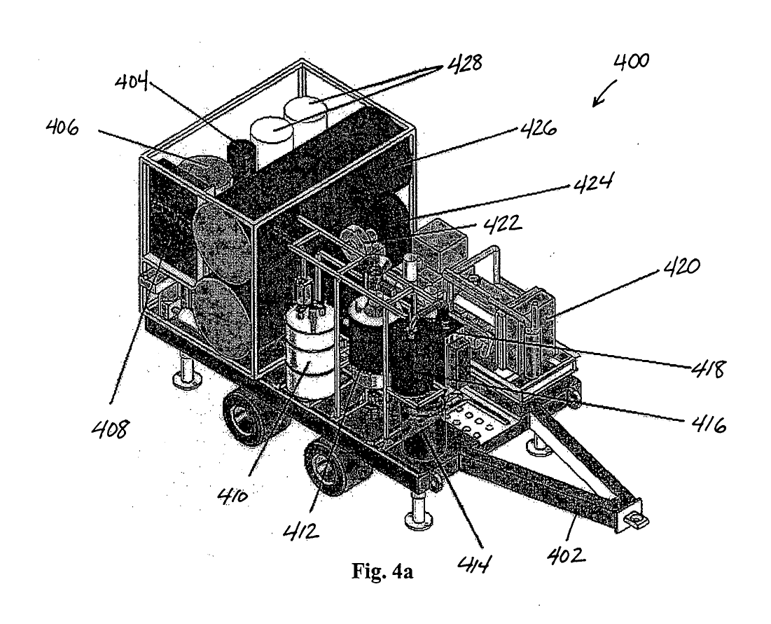

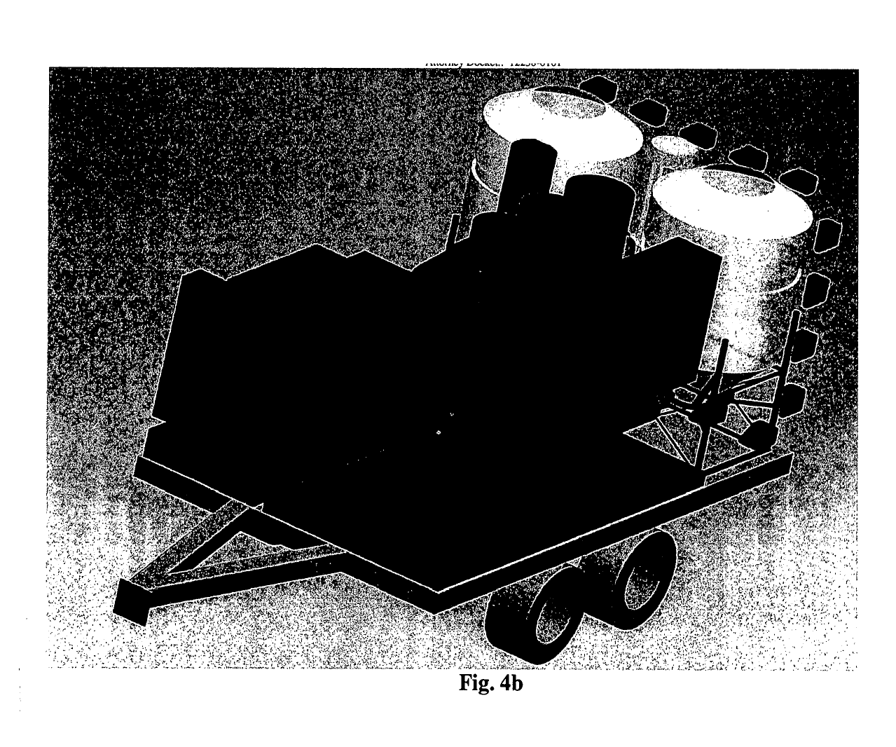

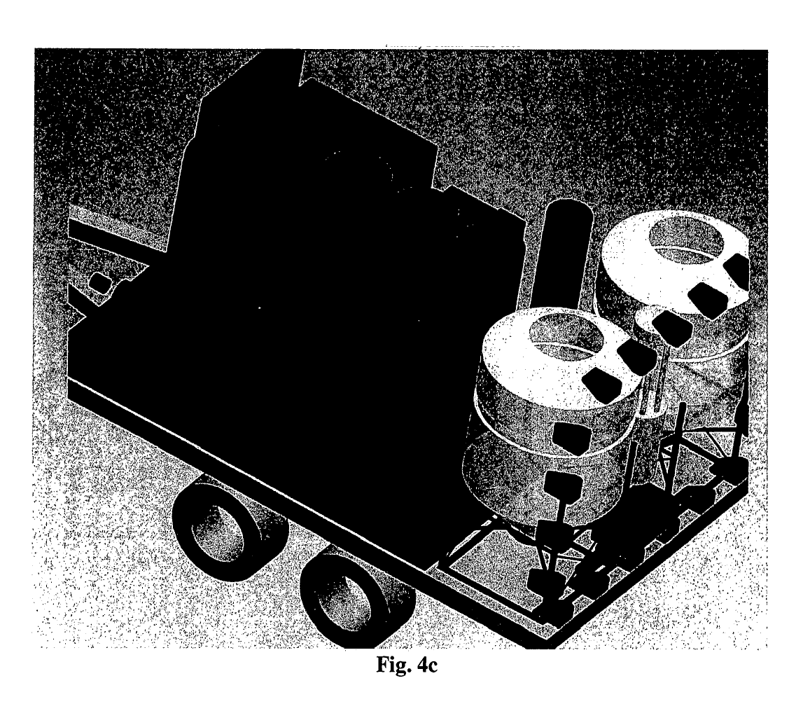

FIGS. 4a-4c illustrate schematically how a biorefinery 400 could be designed and deployed for use to accommodate a waste processing system, such as the V2 Bicameral System, in accordance with the present teachings. Here, the biorefinery 400 is shown deployed on a trailer 402, such as a XM 1048 5-ton trailer. Exemplary biorefineries can comprise a distillation tower 404, a pre-heater 406, controls 408, a filter 410, a gasifier 412, an ash bin 414, a char knock-out pot 416, a heat exchanger 418, an engine/genset 420, a cooling air blower 422, a slow fermentation tank 424, a fast fermentation tank 426 and dehydration chambers 428. A more detailed discussion of the above-referenced components and their operational features is provided throughout the specification with reference to the exemplary processes of the present teachings and does not require further discussion here.

The V2 Bicameral System provides considerable flexibility. For example, if no water source is available for the hydrolysis step, hydrolysis may be bypassed for direct fermentation using excess enzymes and ambient moisture from the wet waste. In addition, output can be adjusted to meet demand by directing more or less input mass to either side. For example, if more ethanol is desired, dry cellulose organics may be directed to the biocatalytic treatment as opposed to the thermochemical treatment.

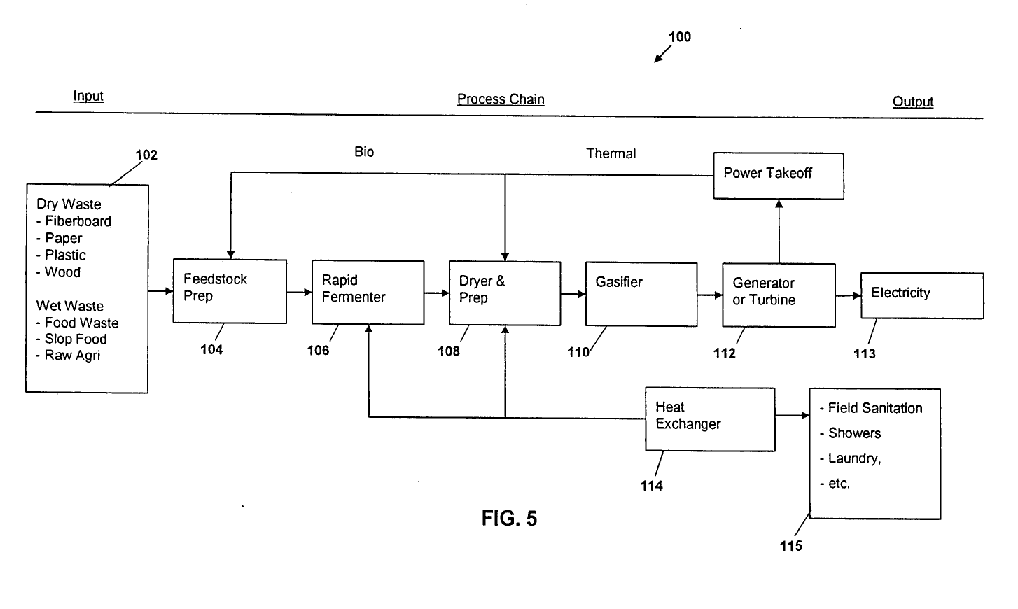

FIG. 5 provides an overview of an in-line integrated thermochemical and biocatalytic energy production system 100. The in-line system 100 allows combined wet and dry waste from a combined source 102 to be delivered to a feed stock preparation station 104. The feed stock preparation station 104 would typically include a grinder for providing a uniform size of organics delivered to the rapid fermenter 106. Output of the rapid fermenter 106 is directed to a dryer and preparation station 108 which may include a distillation column for distilling combined ethanol and water or the combined ethanol and water may simply be delivered to the gasifier 110. Solids from the rapid fermenter would preferably be dried in the dryer and preparation station 108 and in the gasifier 110 organics are converted into methane. The captured methane is provided to a generator or turbine 112 for the production of electricity 113. Electricity 113 may be used at the dryer prep station 108 and with the grinder at the feed stock preparation station 104. Waste heat captured by the gasifier 110 and the turbine generator 112 is provided to a heat exchanger 114 which may be associated with a water supply 115 to provide hot water for field sanitation, showers, laundry and the like and/or may provide heat for the drying and/or distillation at the dryer prep station 108 or for the rapid fermenter 106.

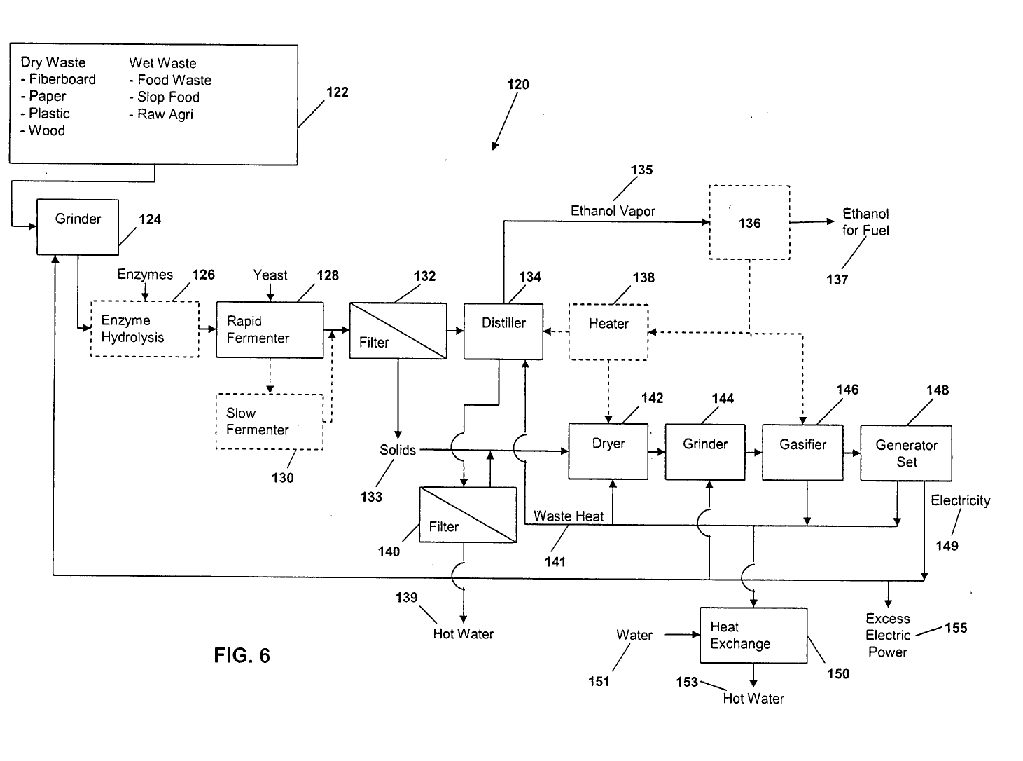

FIG. 6 illustrates in greater detail one embodiment of an in-line integrated thermochemical and biocatalytic energy production system which will be referred to herein as the I1 System 120. In the I1 System 120 combined wet and dry waste from a combined source 122 is delivered to a grinder 124. The waste is ground to a suitable maximum size in the grinder 124 and provided to the hydrolysis chamber 126. The hydrolysis chamber is shown in phantom lines to illustrate that this step may be bypassed by providing enzymes to the rapid fermenter 128 as discussed above with respect to the V2 Bicameral System 50. Where employed, hydrolyzed organics along with the insoluble organics are directed to the rapid fermenter 128 where, in the presence of yeast, soluble organics are fermented into water and ethanol. A slow fermenter 130, shown in phantom lines, is optionally provided to provide further fermentation of the more resistant solubilized organics. Output from the rapid fermenter 128 and, if utilized, the slow fermenter 130, are provided to filter 132 to separate solids 133 from liquids, which will consist primarily of ethanol and water. The liquids are provided to the distiller 134 and subject to distillation. Ethanol vapor 135 from the distiller 134 is provided to an optional condenser 136 which may include means for further removal of any residual water resulting in ethanol output 137 which is suitable for use as a fuel. Alternatively, or in addition, the ethanol vapor 135, before or after condensation, may be provided to heater 138 to provide heat to the distiller 134.

Hot water from the distiller 134 is conveyed to filter 140 with the filtered hot water 139 then being available for field camp uses. Solids from the filter 140 are combined from the filter 132 and conveyed to the dryer 142. Following drying, the solid organics are conveyed to the grinder 144 to be ground to a suitable maximum size. From the grinder 144, the solids proceed to the gasifier 146 for gasification. As with the other embodiments discussed herein, the gasifier may be, for example, a BioMax Unit from Community Power Corporation of Littleton Colo. Methane produced by the gasifier 146 proceeds to the generator set 148 for the production of electricity 149.

As with the V2 Bicameral System 50, the I1 In-line System provides opportunities for capture of waste heat and use of generated electricity to run the system. For example, the ethanol vapor 135 from the distiller 134 may be directed to the gasifier 146 to maximize production of electricity. Waste heat 141 from the generator set 148 and the gasifier 146 may be provided to the dyer 142 and the distiller 134 to aid in these processes. Alternatively, or in addition, the waste heat may be provided to a heat exchanger 150 which may include a water supply 151 to produce hot water 153 for field camp uses or an air supply to provide heated air. Electricity 149 from the generator set 148 can be used to provide the electrical energy requirements of the I1 In-line System. For example, electricity can be provided to the grinder 144 and the grinder 124. Excess electric power 155 can be deployed for other appliances within the field camp.

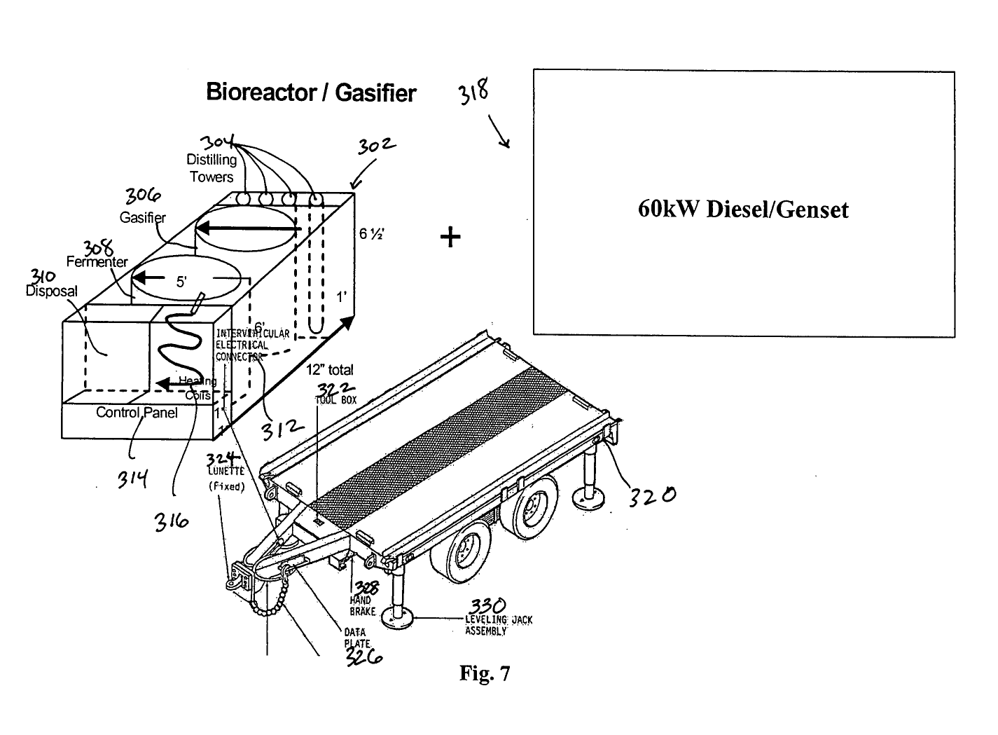

FIG. 7 is an exemplary schematic representation of some of the components of the I1 In-line System and how these components may be deployed on a trailer in accordance with the present teachings. In one embodiment for military applications, the I1 In-line System is designed to accompany a Force Power Module (550 man FPM) and can convert approximately 2,200 pounds of daily mixed waste into 60 kilowatts of electricity, 720 gallons of sterile water and “excess” heat which can be used with heat exchangers to provide hot water for field sanitation, showers or laundry operations. Econometric analysis yielded a daily conservation of 100 gallons of diesel fuel and an aggregate cost savings of approximately $3,800 for each day the I1 In-line System is operated. The cost savings are a product of reduced logistics overhead for the delivery of fuel and the disposal of waste.

In FIG. 7, an exemplary bioreactor/gasifier unit 302 is shown having distilling towers 304, gasifier 306, fermenter 308, disposal unit 310, intervehicular electrical connector 312, control panel 314 and heating coils 316. Gas produced by the gasifier 306 can be fielded as a modification upgrade for the current inventory of a trailer-mounted generator set 318 (such as a 60 kW Tactical Quiet Generator (TQG) supporting the FPM. The unit would be positioned near a Containerized Kitchen system and primarily utilize the waste produced for mess operations, particularly as the 60 kW generator could be configured to connect to the FPM power grid. Here, the bioreactor/gasifier unit 302 is shown deployed on a trailer 320, such as a XM 1048 5-ton trailer. The exemplary trailer is shown having a tool box 322, lunette 324, data plate 326, hand brake 328 and leveling jack assembly 330. A more detailed discussion of the above-referenced components and their operational features is provided throughout the specification with reference to the exemplary processes of the present teachings and does not require further discussion here.

In all embodiments of the integrated thermochemical and biocatalytic energy production system, the gasifier may be “tuned” to a military waste stream and the generator may be a diesel generator modified to transition to both using gas and ethanol added from diesel priming. It is believed that the I1 In-line System, like the V2 Bicameral System, may be operated with nominal training to current FPM generator mechanics and no additional man power will be required. The I1 In-line System is expected to meet all necessary environmental and safety regulations.

While the I1 In-line System and V2 Bicameral Systems are described for use in military operations, these embodiments and all other embodiments of the invention may be used with any suitable commercial, industrial or institutional waste streams including variable amounts of both dry and wet organic wastes. Combining thermochemical and biocatalytic processes as disclosed in the various embodiments allows the overall system to utilize the inherent strength of each technical approach and concurrently mitigate the corresponding limitations of the other. For example, rather than drying and gasifying significant volumes of carbohydrates and sugars resident in wet food waste, it is much more effective to do a rapid fermentation step to produce ethanol and distill it for use in the gasifier or for other uses in the field camp. Extraction of the ethanol in this manner requires less energy input than drying of the food stream that contains both solids and dissolved organics. In addition to transforming the soluble organics into recoverable energy (e.g., ethanol) the biocatalytic process reduces the volume of solids that must be filtered and dried for gasification. Likewise, organics that cannot be efficiently solublized are captured and the residual biomass, dried and subject to thermochemical gasification to extract their energy potential in a more efficient and complimentary manner.

As illustrated by the various embodiments herein, combining biocatalytic and thermochemical processes allows for an exchange of energy and materials between the two subsystems that improves overall system performance. For example, requirements of heat and electricity for the biocatalytic production of food wastes can be provided from the biocatalytic subsystem improving overall system performance. As another example, ethanol produced from the biocatalytic system can be directly introduced either into the gasification module or blended into methane from the gasification module as a vapor to fuel the power generator. The overall result is a self-contained system where a broad range of waste is effectively eliminated via internal exchange of energy and material and an optimal energy output is achieved. The synergistic combination of technologies enables bio-based conversion to be carried out in parts of the world where there is no power grid or external source of energy.

This system enables the wet material to be more efficiently processed (in the field) than if it were dried, size reduced, and then fed to the gasifier directly. The invention provides for sequence of processing steps in which the wet material will be converted to sugars and then to ethanol via a fermentation process. The ethanol is readily separated from the remaining solids through a filtration and distillation process that is described as part of the invention. The hydrous ethanol vapors from the distillation column (approximately 90%) can then be fed to the generator set engine, directly, or mixed with gas from the gasifier and then fed to the generator set (engine).

Advantages and improvements of the apparatuses and methods of the present invention are demonstrated in the following examples. The examples are illustrative only and are not intended to limit or preclude other embodiments of the invention.

### EXAMPLES

### Examples 1-2

Examples 1 and 2 demonstrate the mass balance of food and organic waste into ethanol and/or electricity in accordance with one embodiment of the present teachings.

### Example 1

### (“I1”) In-Line Model

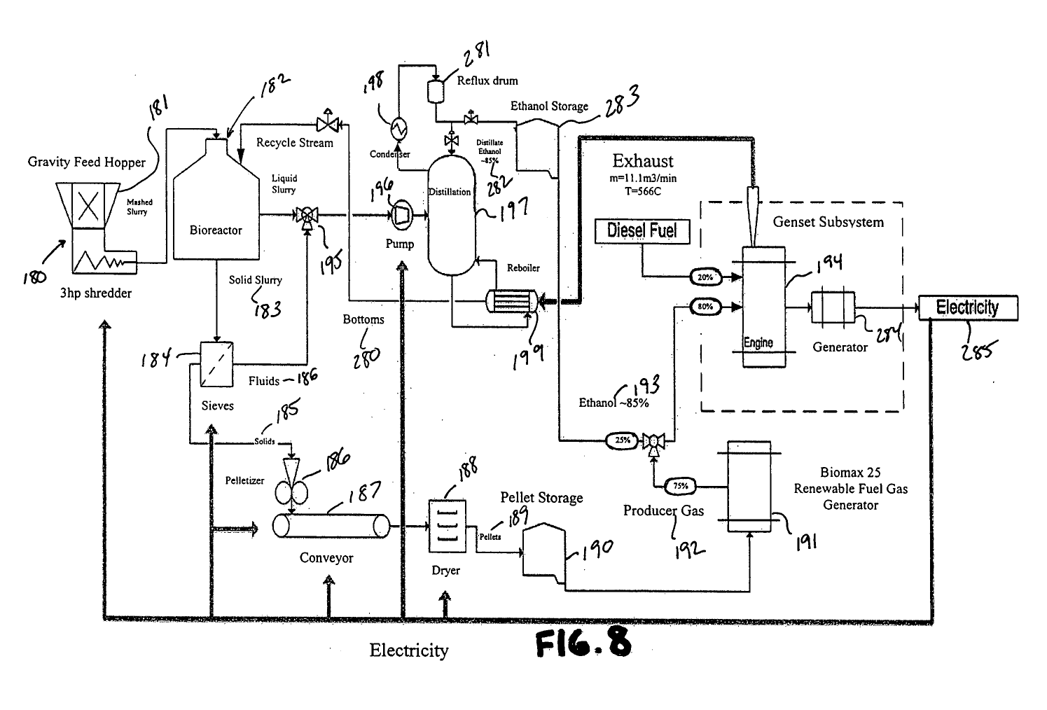

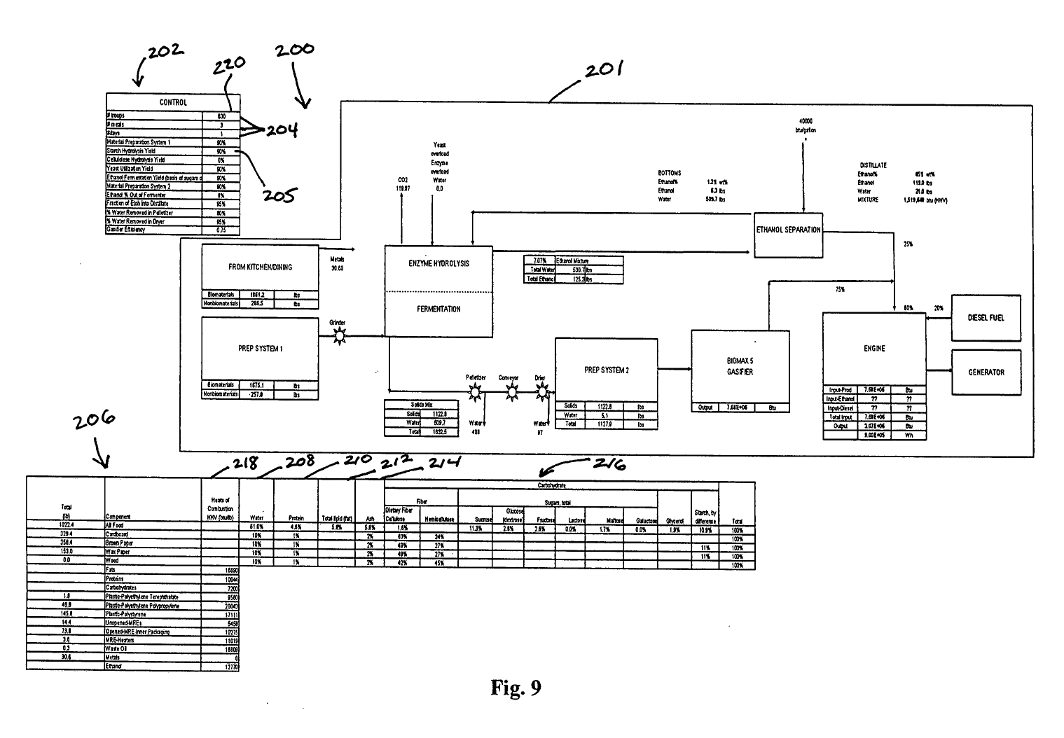

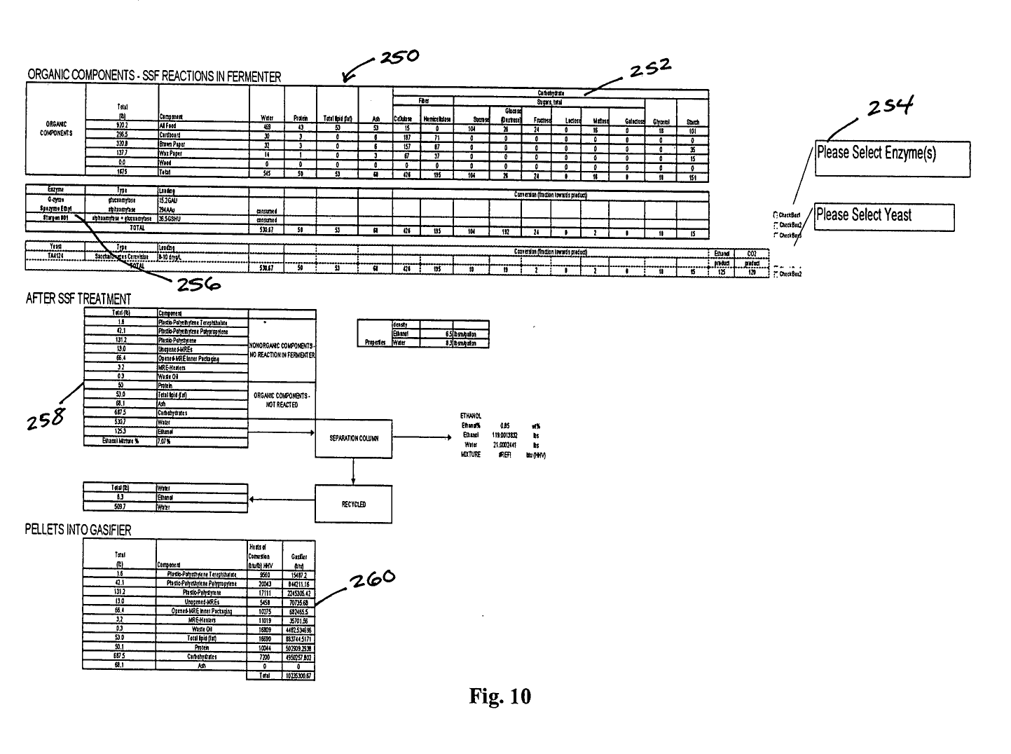

In this Example, waste is subjected to enzyme hydrolysis and yeast fermentation within a bioreactor. The waste has a residence time of less than 24 hrs in the bioreactor after which the ethanol is separated out, and the solids are pelletized. The ethanol and pellets are stored and can be used on demand. The pellets are gasified to make producer gas and mixed with the ethanol for air injection into a diesel engine. A detailed schematic of the process is shown in FIG. 8, while an entire Mathematical Model is shown in FIGS. 9 and 10.

With reference to FIG. 8, wet and dry wastes from a combined source are delivered into a grinder/shredder 180 through a gravity feed hopper 181. While the shredder 180 may operate at different power levels, in this exemplary illustration, the shredder operates at a level of 3 hp. In the shredder 180, the waste is ground to a suitable maximum size before being channeled to the bioreactor 182. Once the materials enter the bioreactor 182, a fermentation process begins whereby the solid materials or solid slurry 183 separates into its components. As the fermentation process is completed, yeast cells settle into the bottom of the bioreactor 182. CO₂ gas is also formed and vented out of the bioreactor through an exhaust duct (not shown). Output from the bioreactor 182 is provided to a filter or sieve 184, which separates solids 185 from liquids/fluids 186. The solids 185 are introduced to a pelletizer device 186 where the solids are pelletized into pellets 189 and carried across a conveyor to a dryer 188. After the pellets 189 are dried by the dryer 188, they can be stored in a storage unit 190 for on-demand use as needed. For instance, the pellets 189 may be gasified by a gasifier 191 to make producer gas 192 and mixed with the ethanol 193 for air injection into a diesel engine 194.

The liquids/fluids 186 are passed through a valve 195 and pumped into a distillation device 197 by pump 196 to undergo a distillation process. Ethanol vapor from the distillation device 197 is provided to a condenser 198, which may include a means for further removal of any residual water resulting in ethanol output which is suitable for use as a fuel. Heat from the distillation process is supplied to a reboiler 199 to generate the vapor. The vapor raised in the reboiler 199 is reintroduced into the distillation device 197 and the liquid removed from the reboiler is known as the bottoms product or simply bottoms 280.

The ethanol vapor moves up a column of the distillation device 197, and as it exits the top of the distiller, it is cooled by the condenser 198. The condensed liquid is then moved to a holding vessel known as the reflex drum 281. Some of the liquid is recycled back to the top of the distillation column and is called “reflux.” The condensed ethanol liquid or distillate 282 that is removed from the system is then stored in an ethanol storage unit 283. The ethanol is distilled into a hydrous form that is suitable for burning in an internal combustion engine 194, or mixed with gas from a gasifier to power the engine used to drive the generator set 284 in order to produce electricity 285.

FIG. 9 depicts an exemplary mathematical model 200 that provides sample data to support the I1 model described in FIG. 8. It is initially noted that the schematic flow diagram shown within box 201 of FIG. 9 operationally corresponds to the process described in detail above with reference to FIG. 8 and therefore does not require additional discussion at this point. Moreover, the data shown within box 201 is merely provided as an exemplary illustration of how the I1 model can be implemented and mathematically calculated in accordance with the present teachings. As such, the present example is not intended to be limiting in scope herein.

The first notable box of the mathematical model 200 is the CONTROL box 202, which also corresponds to Table A, below. The first three rows 204 are for inputting the total meals served, the current setting being set for 600 troops, 3 meals a day, for 1 day. Based on this information, the Total (Ib) of each waste component is listed in materials table 206. The materials table 206 has pre-set composition data for water 208, protein 210, fat 212, ash 214, and carbohydrate content 216, as well as heats of combustion 218. The amount of each constituent, type of constituent, etc. can be changed in the model at any time.

The CONTROL box 202 also has inputs for unit operations and yield coefficients 220 for beginning to end reaction conversion efficiencies. The current setting assumes 90% material makes it through the material prep systems, 90% of total starch is converted to glucose, 90% of sugars are utilized for product formation as opposed to cell growth, and 90% of potential ethanol yield is realized. The setting also includes the concentration of ethanol out of the fermenter and the fraction of ethanol desired in distillate, set at 8% (80 g/L ethanol) and 95% (850 g/L), respectively.

For the solids portion of the model, the CONTROL box 202 has inputs for % water removed in pelletizer, % water removed in dryer, and gasifier efficiency; 80%, 95%, and 0.75, respectively. All of these values can be changed to represent a more accurate scenario or to predict the influence of system changes if needed.

TABLE A
Constants for Waste Stream Conversion
CONTROL

| | |
|---|---|
| # troups | 600 |
| # meals | 3 |
| #days | 1 |
| Material Preparation System 1 | 90% |
| Starch Hydrolysis Yield | 90% |
| Cellulolose Hydrolysis Yield | 0% |
| Yeast Utilization Yield | 90% |
| Ethanol Fermentation Yield | 90% |
| Material Preparation System 2 | 90% |
| Ethanol % Out of Fermenter | 8% |
| Fraction of Ethanol into Distillate | 95% |
| % Water Removed in Pelletizer | 80% |
| % Water Removed in Dryer | 95% |
| Gasifier Efficiency | 0.75 |

In the section labeled “ORGANIC COMPONENTS-SSF REACTIONS IN FERMENTER” 250 in FIG. 10, the total amount of each carbohydrate component 252 is displayed and with this, the amount of sugars released and the amount of ethanol produced is calculated. Enzymes can vary significantly, therefore, to account for the possibility of using a mixture of enzymes, each with a specific activity; the model has been set up to allow the “choice” of enzymes 254. In this example, the enzyme chosen is Genencor's Stargen 001 256. Stargen is a low temperature alpha- and glucoamylase mixed enzyme product with the ability to readily break down starch and maltose. The theoretical hydrolysis conversions for starch, cellulose, and maltose into glucose and xylan into xylose are shown together with their equations below. In the model, the theoretical conversion is multiplied by the “Starch Hydrolysis Yield,” listed in Table A above (see also reference numeral 205 in FIG. 9), to determine actual conversion.

To calculate the theoretical conversion of polysaccharides such as starch (S) or cellulose (C) into glucose (G), the reaction of 1 mole anhydroglucose unit (MW=162) with water to form 1 mole glucose (MW=180) is calculated on a mass basis by multiplying C+S by 180/162. For disaccharides such as sucrose (U) and maltose (M), the conversion of 1 mole disaccharide (MW=342) into 2 mole (2 glucose for maltose and 1 glucose for sucrose) is calculated by multiplying ½U+M by 180(2)/342. The theoretical conversion (100% hydrolysis) of polysaccharides and disaccharides into glucose is thus:

$$G = {G}_{0} + ( C + S ) \frac{180}{162} + ( \frac{1}{2} U + M ) \frac{( 2 ) 180}{342}$$

Using the molecular weights and mole balances of the hydrolysis reaction equations, the total theoretical conversion into glucose, fructose and xylose and the total theoretical consumption of water can be expressed as the equations:

$$F = {F}_{0} + ( \frac{1}{2} U ) \frac{( 2 ) 180}{342} ;$$

$$Water Consumption = ( C + S ) \frac{18}{162} + ( U + M ) \frac{( 2 ) 18}{342}$$

Cellulose conversion will only be calculated if cellulases are used and the cellulosic hydrolysis yield and starch hydrolysis yield can be set differently in Table A. Xylan (hemicellulose) will not be present in significant quantities. In addition, the yeast can not ferment xylose. As a consequence, there is not much incentive to break down the xylan to xylose. It is, however, included in this model as those skilled in the art will appreciate that it may have significant uses in other exemplary models or variations of the present teachings.

For fermentation, the yeast will consume glucose before other hexose sugars. By the end of the fermentation it will consume all hexoses and produce ethanol, CO₂, and other byproducts. The reaction for hexose into ethanol and CO₂ is displayed below. For each mole of hexose consumed, the yeast produces 2 moles ethanol and 2 moles CO₂. Ethanol production competes with glycerol production. Each mole of hexose can also produce 2 moles of glycerol. This does not include yeast utilization yield and ethanol fermentation yield which is the fraction consumed towards ethanol production and the yield of fermentation itself. Both constants are in listed in Table A.

Theoretical conversion of hexose (H) (glucose (G) or fructose (F)) into ethanol (E), glycerol (L) and CO₂, assuming no initial ethanol present:

$$E = ( H ) \frac{( 2 ) 46}{180} = ( H ) [ 0.511 ] ;$$

$$L = ( H ) \frac{( 2 ) 92}{180} = ( H ) [ 1.022 ] ;$$

$${CO}_{2} = ( H ) \frac{( 2 ) 44}{180} = ( H ) [ 0.489 ] .$$

Once the ethanol conversion from carbohydrates is subtracted from the mix, the remaining proteins, lipids, and carbohydrates from the biomaterials stream and the amounts of non-biomaterials are combined, dried, and pelletized. Water removal conversions for each step of the pelletizing process are in Table A. In the current model, 140 lbs 85% wt ethanol and 1128 lbs pellets are formed per day. Based on the contents of the pellets and mass of ethanol, the btu's generated by the heat unit are calculated by multiplying Heat of Combustion (HHV) by mass. The temperature of the gasified material is not taken into consideration for the current model, but may be in the future. For now, heats of reaction determined in the Ft. Polk study at standard temperature pressure (STP) were used:

$$Δ H ( btu ) = conversion * \sum ( component mass ( lb ) * HHV ( \frac{btu}{lb} ) )$$

A breakdown of the material components after they have undergone SSF treatment according to this exemplary model is shown in table 258, while the breakdown of the pellets delivered to the gasifier is shown in table 260.

### Example 2

### Segregated System (“S Model”)

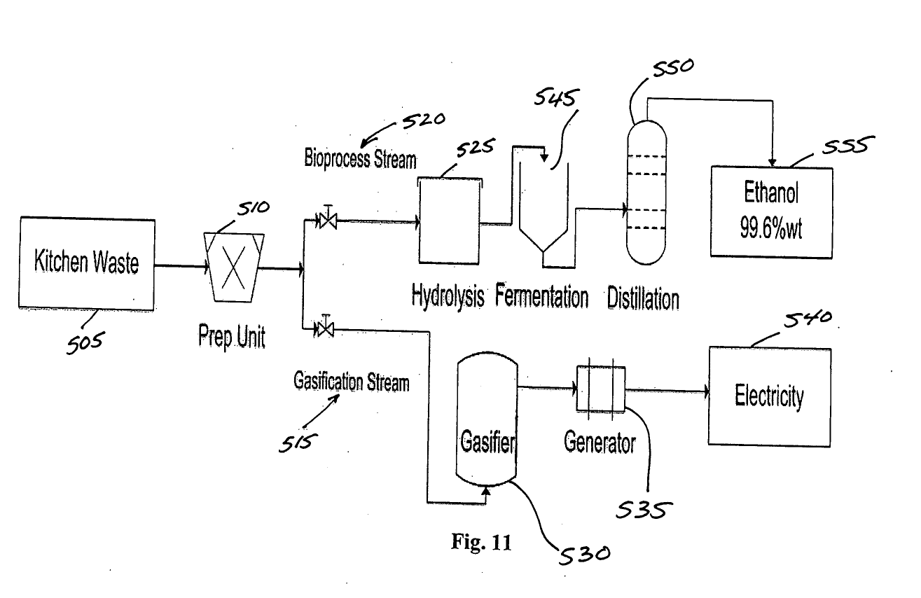

In this model (referred to as the “Segregated System” or “S Model”), waste is separated and run in segregated systems known as the “Segregated System” and is shown in FIG. 11. The “Segregated System or “S Model” was used in an exercise to determine the potential for generating electricity and/or ethanol from Purdue Yard and Cafeteria Waste. The I1 is described for 600 troops per day capacity and the S Model is described for yearly waste produced at Purdue University Campus.

All pruning, mowing, mulching, tree trimming and pickup of leaf and brush waste is directed by the Purdue Grounds Department located in the Building Services and Grounds Building. Grounds clippings and leaves are removed through two types of pickup methods (1) Service Trucks for brush and (2) Vacuum Trucks for leaves for a total of 297,000 cubic feet brush/year and 105,000 cubic feet brush/year. Service Trucks and Vacuum Trucks dispose of brush and leaves into a holding area in a gravel pit just south of the Building Services and Grounds Building. They are then transferred to an independent soil composting program near campus.

Currently, Purdue University composts yard waste and pays a tipping fee for cafeteria waste removal. The cafeteria waste from residence halls on campus is collected off of conveyer belts in the kitchen and then run through a pulper and centrifuge which grinds the food and removes a majority of the water. Workers are discouraged from allowing nonfood items into the waste, so it is close to 100% food waste. If approximately 9,200 lbs per week food is collected from campus cafeterias and if there is potential to collect an additional 8,000 lbs per week, this would lead to 17,200 lbs per week if all cafeterias participate.

The amount of wet and dry yard and food waste generated on a university campus each year is listed in Table 2-A below. The wet weight would be calculated as 17,200 lbs per week for 40 weeks (summer operation is not included). The density of the food waste is from an Army study at Fort Polk in 2000. The leaves and brush density were measured roughly outside and are estimated at 5 and 1 lbs/ft³, respectively. This would lead to 500,000 lbs dry yard waste and 206,000 lbs dry food waste per year.

TABLE 2-A
Dry Mass of Yard and Food Waste Generated at Purdue per Year

| | | | | | % Water | | |
|---|---|---|---|---|---|---|---|
| | | | | | Weight | | |
| | | | | | Fraction | | |
| | | Volume | Density | Biomass Wet | Content of | Biomass Dry | Total Dry |
| Type | | (ft\hat{ }3) | (lbs/ft\hat{ }3) | Weight (lbs) | Biomass | Weight (lbs) | Weight (lbs) |
| Yard | Leaves | 10500 | 5 | 525000 | 0.5 | 262500 | 500100 |
| Waste | Brush | 297000 | 1 | 297000 | 0.2 | 237600 | |

| | | | | | | |
|---|---|---|---|---|---|---|
| Food Waste | 76444 | 9 | 688000 | 0.7 | 206400 | 206400 |

The constituents (ash, hemicellulose, cellulose, etc.) and heat of combustion for each component are given in Table 2-B below. Material constituents for leaf and brush waste are estimated for Xylan, Ash, and Protein content and are within ranges given for typical hemicellulose and cellulose composition in deciduous trees and food waste is estimated as high in starch, fat, and sugar. Heats of combustion were obtained from the Fort Polk Army study.

TABLE 2-B
Constituents and Heats of Combustion Used in Segregated Model

| | | | | | | | | Heat of |
|---|---|---|---|---|---|---|---|---|
| | Material Constituents - Dry Basis (%) | | | | | | | Combustion Wet |
| Type | Starch | Cellulose | Sugar | Hemicellulose | Xylan | Ash | Fat, Protein | Basis (Btu/lb) |
| Food Waste | 22.3% | 3.3% | 41.4% | | | 11.8% | 21.2% | 2370 |
| Yard Waste | | 22.0% | | 68.0% | 7.0% | 2.0% | 1.0% | 8189 |

With reference to FIG. 11, kitchen waste 505 (i.e., wet and dry yard and food waste) is introduced to a feed preparation unit 510, which may include a grinder to break the dry waste into adequate size for treatment in the subsequent gasifier unit 530 and/or a dryer (not shown) to remove excess moisture content. From the feedstock preparation unit 510, the dry waste is subjected to a gasification stream 515 and is fed to the gasifier unit 530 where it undergoes a thermochemical treatment process for the production of a methane containing gas. The methane gas is conveyed to a generator 535 for the production of electricity 540. Waste heat from the thermochemical reactions of the gasifier unit 530 or the generator 535 may be used for the production of hot water or in the biocatalytic process.

The biocatalytic process begins with the feed preparation unit 510 dissolving or hydrating the biomass wastes so that enzymes can more easily hydrolyze the wastes. As stated above, the preparation unit 510 may include a grinder for breaking or pulverizing solubilizable material into smaller particles for improved solubilization. After the waste leaves the preparation unit 510 and continues down the bioprocessing stream 520, the waste is subjected to a hydrolysis chamber 525 for thermal or enzyme hydrolyzation of the wet waste. The wet waste is then delivered to a fermentation unit 545 where in the presence of yeast, the solubilized organics are fermented into ethanol and water. Where appropriate, enzymes may be provided to the fermentation unit 545 for simultaneous hydrolyzation and fermentation of the wet organic waste. The addition of enzymes assists in converting starch or cellulose into monosaccharides (glucose or pentose). Following fermentation, the recovered mixture of ethanol and water is separated by, for example, distillation in a distillation unit or column 550. In this exemplary illustration, the ethanol separation process is completed at 99.6% 555.

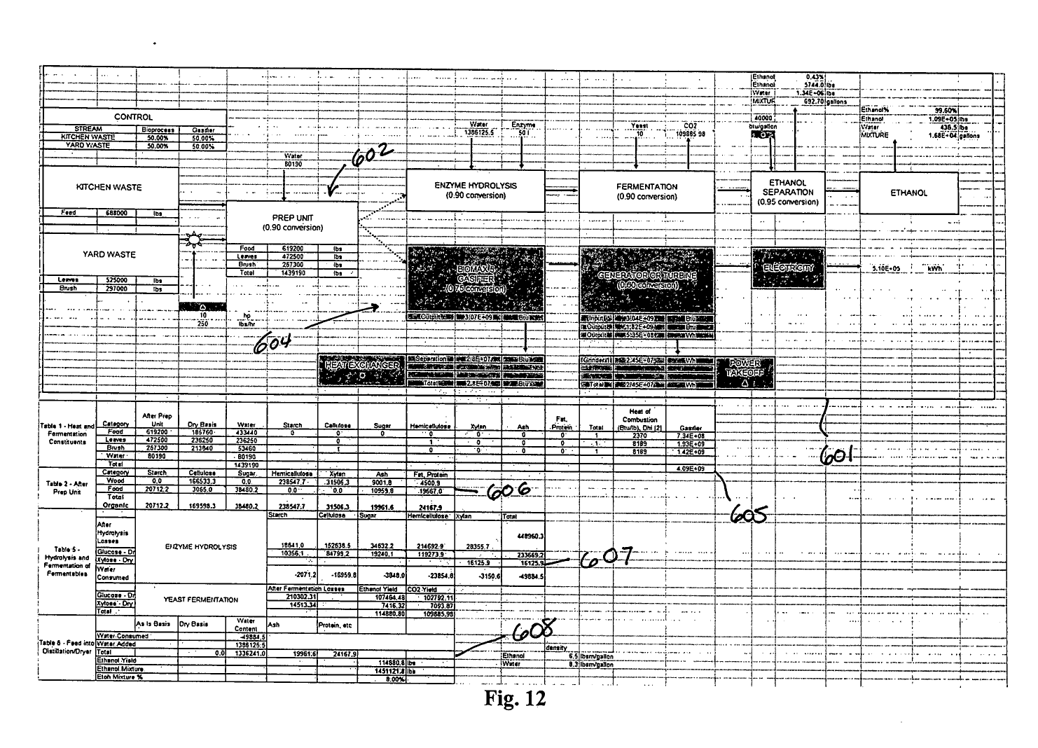

An exemplary mathematical model supporting the above process is shown in FIG. 12. It is initially noted that the schematic flow diagram shown within box 601 of FIG. 12 operationally corresponds to the process described in detail above with reference to FIG. 11 and therefore does not require additional discussion at this point. Moreover, the data shown within box 601, as well as Tables 1, 2, 5 and 6 (identified by reference numerals 605, 606, 607 and 608 respectively), are merely provided as an exemplary illustration of how the S model can be implemented and mathematically calculated in accordance with the present teachings. As such, the present example is not intended to be limiting in scope herein.

The waste can go into either the bioprocessing side 602 to form ethanol (no shading) or the gasification side 604 to form electricity (grey). The amounts of waste into each can be controlled depending on the desired outcome. The inputs listed in FIG. 12 are for the yearly amount of waste produced by Purdue for both food and yard waste going 50% into each process. These values represent accumulated output for the whole year. In reality, the capacity size of the equipment as well as the operation and waste storage will be limited to daily, weekly, or monthly operation. In the segregated model, ethanol separation is completed to 99.6%. To calculate the gallons of ethanol for a particular % ethanol mix, where ρₑₜₕₐₙₒₗ=6.5 lbsm/gallon and ρ_{water}=8.3 lbsm/gallon:

$$Ethanol ( gal ) at • \% = \frac{Ethanol ( lbs )}{• * {ρ}_{ethanol} ( lb / gal ) + ( 1 - • * {ρ}_{water} ) ( lb / gal )}$$

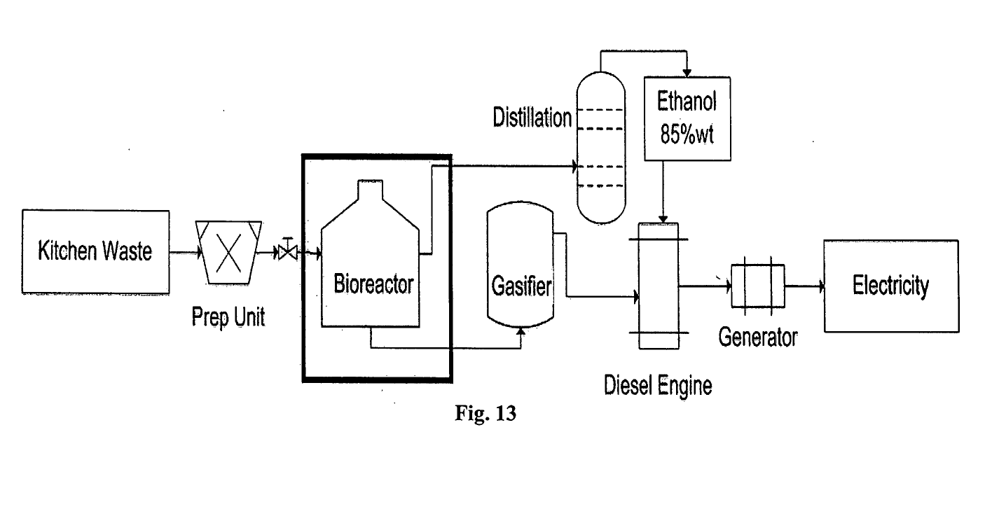

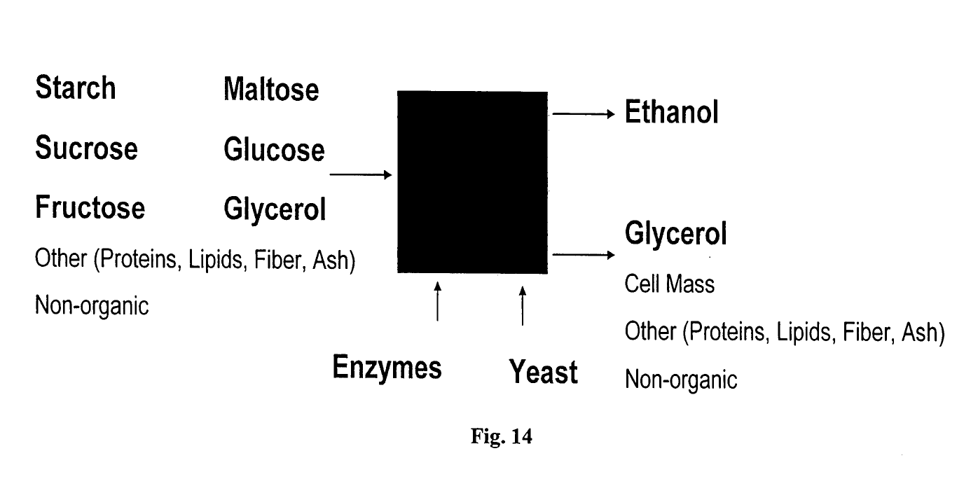

The mathematical Inline and Segregated Models of Examples 1 and 2 use simple mass balances with constant parameters to determine the final concentration of solids and ethanol. If average conversion efficiencies and yields are known for a particular enzyme/yeast combination at a certain temperature and time period then this model could be simple and useful. However, if different enzyme/yeast combinations and varying temperatures are a possibility, then a robust model which can account for these variances is needed. A model which can predict how the concentrations are changing over time, and how much enzyme and yeast to add, will result in optimal ethanol production with the least amount of resources and energy needed. The bioreactor sugar inputs and the ethanol and glycerol outputs are modeled as ordinary differential equations. The Bioreactor portion of the Inline model design with all waste material entering and ethanol and solids leaving can be drawn as a black box as seen in FIG. 13 with inputs and outputs displayed in FIG. 14.

### Examples 3-5

Examples 3-5 illustrate material and energy balances in accordance with the processes of the present teachings. The processing steps require experimental verification, and this is provided through the examples for inline processing where a simulated mixture of solids and food waste are processed through fermentation and the unfermented solids recovered, filtered and made into pellets. For clarity purposes, the models are described below as versions V1, V2 and V3

### Example 3

### In-Line System Calculation—Kitchen Waste from 600 Troops (V1)

This model was developed to carry out a material and energy balance analysis. The method of calculating the material balance is to use algebraic equations taking into account the weights of materials as inputs and multiplying them by the fractional compositions as indicated in the input tables provided below. Table 3 also includes heats of combustion that are related to the energy value of the various components, as well as conversion factors that are used in performing these equations. Calculations are carried out by referring to the components that contain values of conversion factors or compositions and multiplying, adding, etc in the appropriate manner. Calculated results for several cases using this exemplary process are summarized in Table 4.

In this example, an in-line integrated thermochemical and biocatalytic system for processing kitchen waste (e.g., cardboard, paper and oil) in accordance with the present teachings is shown. This model takes the input material from wastes generated by 600 troops and utilizes the composition of the kitchen wastes, with their properties summarized in Table 1 below, and the weights of the various streams generated in Table 2. The output of the bioreactor is described in terms of ethanol at a 99.6% basis, and as a dilute ethanol (0.38%) obtained from the distillation column. While distillation and drying is carried out in this example, the ability of the internal combustion engine to take 85% vaporous ethanol as a feed obviates the need for carrying out the drying step. Distillation to 85% is sufficient, thus avoiding the extra capital and operating cost of drying the ethanol and bypassing the azeotrope.

The model itself shows how the various streams are handled. The boxes are shown for clarity so that the materials flows are readily apparent. However, multiple unit operations, for example, separation, hydrolysis, and fermentation may occur in one vessel, thus simplifying the mechanical design of the bioreactor. This bioreactor is referred to as a fast bioreactor since conditions are chosen so that there are high levels of enzyme and yeast to rapidly achieve the production of ethanol and the separations of solid components (they float). The liquid fermentation broth contains ethanol, while the solids are physically separated. The liquid is processed separately in a distillation column, and the solids are pelletized separately.

The model shows the ethanol as a separate stream. It should be noted that if the ethanol were 99.6% (essentially water free), it could be used as a transportation fuel. However, the small generators currently used by the Army require JP 8 (a form of kerosene) and hence ethanol may not be suitable. The ethanol could be mixed with gasoline to operate gasoline powered engines. A preferred use is to combine the ethanol with producer gas to provide fuel for the engine that generates electricity. These combinations are not shown in the model. Rather the model presents an example mass and the energy balance of the various streams to indicate how much energy could be available to generate electricity.

The V1 model assumes that all of the cellulose and starch is converted to glucose and the glucose is then fermented. The total mass of kitchen waste is 2178 lbs (dry weight basis). This is based on two MRE's (“Meals Ready to Eat”) and 1 hot meal per day, which results in the composition of waste given in Table 3. For the purposes of this calculation, all of the cardboard (cellulose) is assumed to go to the bioprocessing step. Since the glucose that would potentially be available is much higher than if a major part of the cardboard went to the gasifier (this is the case that was calculated for V2—800 troops in Example 4 below), the ethanol yield at 51.8 gal per day is also higher than the case for V2 where the ethanol is 33.5 gallons per day because 77% of the material goes to the drier. The amount of ethanol that could be produced is not a linear function of the cardboard diverted to the bioreactor, since part of the glucose is obtained from the starch and sugar content in the kitchen waste, all of which goes to the bioreactor (in this Example a BioMax 5® bioreactor model), and more of which is generated for the case of 800 people compared to 600 people.

Other assumptions according to this model include: (1) the design input for the BioMax 5® is 14.3 lbs/hr (dry basis) or 15.7 lbs/hr (at 10% moisture). In order to meet this constraint, all of the cardboard is fed to the bioprocessing section of the tactical refinery. This requires that cellulase enzymes, which hydrolyze cellulose, be used to carry out hydrolysis in addition to amylases, which hydrolyze starch, and that the bioreactor volume be designed accordingly; (2) the efficiency of conversion of the wax paper, plastic and other residual material is assumed to be 55%; and (3) the organic lignin in the cardboard is assumed to not be hydrolyzed by enzymes and will be recycled back to the BioMax 5®.

TABLE 1
Number of troops 600
Conditions

| | | |
|---|---|---|
| Moisture in food | 50% | Xw |
| Moisture in | 10% | |
| Paper/plastic | | |
| Moisture in slop | 90% | |
| Density, ethanol, 20 C | 0.78 | g/mL |
| Density, water 20 C | 1.00 | g/mL |
| L/gal | 3.78 | |

Conversion and Recovery Factors

| | | |
|---|---|---|
| Separation Ethanol | 0.95 | Ys |
| Enzyme Reaction | 0.9 | Ye |
| Distillation Energy | 20,000 | Btu/gal |
| Ethanol Drying | | |
| Energy | 10,000 | Btu/gal |
| Density 99.6% | | lbs/gal |
| Ethanol | 6.6 | est |
| Fermentation | 0.900 | Yf |
| Distillation and | 0.950 | Ym |
| drying | | |
| Gasifier to Heat | 0.550 | Yg |
| Gasifier to Electricity | 0.207 | Yl |
| Delta H Water | 1000 | btu/lb |
| % Water recycle | 65% | |
| lbs/gal water | 8.34 | |

TABLE 2
Weights of materials generated by 600 troops.
Materials

| | | | Units |
|---|---|---|---|
| Input | | | |
| Kitchen Waste | | 2178.2 | lb |
| Kitchen Oil | | 0.2 | lb |
| Enzyme | | 40.0 | L |
| Yeast | | 8.0 | lb |
| Water | | 3987.4 | lb |
| Output | | | |
| Metals | | 46.2 | lb |
| CO2 | | 344.5 | lb |
| Eth/H2O | 0.38% | 54.8 | gallons |
| Solids | | | lb |
| Ethanol | 99.60% | 51.8 | gallons |

TABLE 3
Composition of waste
for VI - 600 person case

| | Weight Per | | | | | | | | | | |
|---|---|---|---|---|---|---|---|---|---|---|---|
| | Person Per | Weight Per | | | | | | | | | Gasification |
| | Meal | day for 600 | | | | | | | | | Heat of |
| | (lb/(person * | troops, 3 | Constituents for Fermentation Calculations | | | | | | | | Combustion |
| | meal)) | meals/day, | | | | Hemi- | | | | | (Btu/lb), Dhi |
| Category | [2] | Xi | Starch | Cellulose | Sugar | cellulose | Xylan | Ash | Fat, Protein | Total | [2] |
| Cardboard | 0.183 | 329.40 | | 73.00% | | 17.00% | 7.00% | 2.00% | 1.00% | 100.00% | 7370 |
| Food | 0.317 | 570.60 | 60.00% | | 30.00% | | 3.00% | 2.00% | 5.00% | 100.00% | 2370 |
| Slop Food | 0.251 | 451.80 | 60.00% | | 30.00% | | 3.00% | 2.00% | 5.00% | 100.00% | 1000 |
| Metal-Aluminum | 0.003 | 5.40 | | | | | | | | | 0 |
| Metal-Iron | 0.014 | 25.20 | | | | | | | | | 0 |
| Paper-Brown | 0.198 | 356.40 | 11.00% | 59.00% | | 20.00% | 7.00% | 2.00% | 1.00% | 100.00% | 7370 |
| Paper-Wax | 0.085 | 153.00 | 11.00% | 59.00% | | 20.00% | 7.00% | 2.00% | 1.00% | 100.00% | 9267 |
| Plastic- | 0.001 | 1.80 | | | | | | | | | 9560 |
| Polyethylene | | | | | | | | | | | |
| Terephthalate | | | | | | | | | | | |
| Plastic- | 0.026 | 46.80 | | | | | | | | | 20043 |
| Polyethylene | | | | | | | | | | | |
| Polypropylene | | | | | | | | | | | |
| Plastic- | 0.081 | 145.80 | | | | | | | | | 17111 |
| Polystyrene | | | | | | | | | | | |
| Unopened-MREs | 0.008 | 14.40 | | | | | | | | | 5458 |
| Wood | 0 | 0.00 | 0.00% | 52.00% | | 38.00% | 7.00% | 2.00% | 1.00% | 100.00% | 8189 |
| Opened-MRE | 0.041 | 73.80 | | | | | | | | | 10275 |
| Inner Packaging | | | | | | | | | | | |
| MRE-Heaters | 0.002 | 3.60 | | | | | | | | | 11019 |
| Waste Oil | 1.65E−04 | 0.20 | | | | | | | | | 16809 |

| | | | |
|---|---|---|---|
| Total | 2178.20 | Separated Fats, | 10,000 |
| | | Proteins | |

TABLE 4

| Description | Input | Output |
|---|---|---|
| Kitchen waste, some cardboard to | 1746 | 35 |
| bioprocessing, lbs/day | | |
| Cardboard, paper, plastic to BioMax 15, | 328 | negl |
| lbs/day | | |
| Kitchen oil, protein, fat, lbs/day | 13.2 | 0 |
| Metals, lbs/day | 46.2 | 46.2 |
| Enzyme, L/day | 40 | 40 |
| Yeast, lbs/day | 8 | >8 |
| Added Water, at 65% recycle (lbs per | (925/111) | (925/111) |
| day/gal per day) | | |
| BioMax Grinder, Kw | 3.8 | 0 |
| | (1 hrs, 5 Hp) | |
| Bioprocess macerator, Kw | 0.37 | 0 |
| | (1 hr, 0.5 HP) | |
| Process Heat Energy, Btu/hr | (propane) | 65,000 |
| Use for distillation | | (64,800) |
| Use for heating water | | (200) |
| BioMax “Waste Heat”, Btu/hr | 0 | 46,400 |
| Electrical Energy², Kw | | |
| Average | 0 | 4.8 |
| Minimum | 0 | 0.9 |
| Ethanol lbs/hr | 0 | 14.3 |
| Gal/hr | 0 | 2.2 |

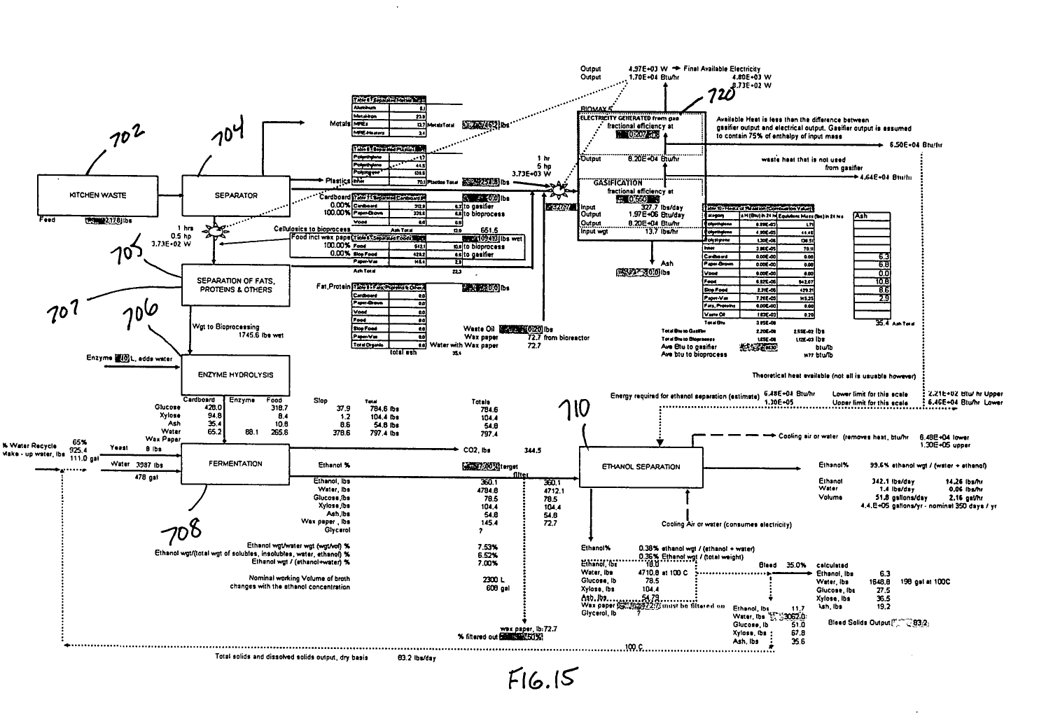

Referring now to FIG. 15, a detailed schematic and mathematical model supporting the data included in the above tables is provided. According to this schematic, kitchen waste 702 (as detailed in Table 3 above) is delivered to a separator 704 that removes metals, plastics and cardboard. The metal is separated out so that it does not enter the gasification unit 720. The other waste materials pass through a 5 HP grinder (such as the BioMax 5® unit) and are ground to a suitable size for introduction into the gasification unit 720. After the waste is ground, they enter the gasifier and a producer gas (low in nitrogen) is made. This gas then goes to the engine/generator set (not shown separately in this diagram) and is used to generate electricity. Some of the electricity is needed to run the rest of the system, while the remaining power available for other sources.

The other wastes that have been separated out by the separator 704 (e.g., uneaten food, fats, oils and paper wastes) is passed through a 0.5 HP grinder 705, which is powered by electricity generated through the biorefinery. At start-up, JP 8 or gasoline can be used to ignite the internal combustion engine that powers the generator set. Once the process begins, the fuel will be cut back and supplanted by the gas stream from the gasification unit 720.

After the additional wastes pass through the second grinder 705, the fats and oils are separated by flotation and they too are skimmed off and sent to the gasification unit 720. Next, the wastes undergo a hydrolysis process 706, in which enzymes are added to break down the starch and other material (e.g., cellulose in card board for example) to glucose. Finally, the glucose is fermented to ethanol in a fermentation step 708.

The fermentation broth that contains ethanol is sent to a distillation column 710 and separated into an ethanol rich phase which is subsequently dried using a molecular sieve. A water rich phase also occurs in which a small amount of ethanol comes out of the bottom of the distillation column 710.

In alternative embodiments according to this exemplary example, an inline system can be constructed wherein wastes are separated and fermented within the same vessel, with the bioreactor and fermentation resulting in the separation (by flotation and settling) of solid materials. These materials are then collected and formed into pellets. The pellets are required by the gasification unit for the purposes of controlling the reaction, and being able to have the right size and density for metering the solids into the gasifier.

### Example 4

### In-Line System Calculation—Kitchen Waste from 800 Troops (V2)

This model repeats the calculations of Example 3 but analyzes 800 troops rather than 600. The calculations are the same as Ex. 3 except that the total mass of generated waste is 2904 lbs (dry basis) and 77% of cardboard is assumed to be diverted to the gasifier, instead of 0% as was the case for the 600 troop analysis. As in Example 3, the heats of combustion that are related to the energy value of the various components, as well as conversion factors that are used in performing these equations are included (see Table 3 below), while the calculated results for several cases using this exemplary process are also provided (see Table 4 below).

In this example, an in-line integrated thermochemical and biocatalytic system for processing kitchen waste (e.g., cardboard, paper and oil) in accordance with the present teachings is shown. This model takes the input material from wastes generated by 800 troops and utilizes the composition of the kitchen wastes, with their properties summarized in Table 1 below, and the weights of the various streams generated in Table 2. The output of the bioreactor is described in terms of ethanol at a 99.6% basis, and as a dilute ethanol (0.38%) obtained from the distillation column. While distillation and drying is carried out in this example, the ability of the internal combustion engine to take 85% vaporous ethanol as a feed obviates the need for carrying out the drying step. Distillation to 85% is sufficient, thus avoiding the extra capital and operating cost of drying the ethanol and bypassing the azeotrope.

The model itself shows how the various streams are handled. The boxes are shown for clarity so that the materials flows are readily apparent. However, multiple unit operations, for example, separation, hydrolysis, and fermentation may occur in one vessel, thus simplifying the mechanical design of the bioreactor. This bioreactor is referred to as a fast bioreactor since conditions are chosen so that there are high levels of enzyme and yeast to rapidly achieve the production of ethanol and the separations of solid components (they float). The liquid fermentation broth contains ethanol, while the solids are physically separated. The liquid is processed separately in a distillation column, and the solids are pelletized separately.

The model shows the ethanol as a separate stream. It should be noted that if the ethanol were 99.6% (essentially water free), it could be used as a transportation fuel. However, the small generators currently used by the Army require JP 8 (a form of kerosene) and hence ethanol may not be suitable. The ethanol could be mixed with gasoline to operate gasoline powered engines. A preferred use is to combine the ethanol with producer gas to provide fuel for the engine that generates electricity. These combinations are not shown in the model. Rather the model present an example mass and the energy balance of the various streams to indicate how much energy could be available to generate electricity.

The V2 model assumes the following factors: (1) The design input for the BioMax 15® is 42 lbs/hr (dry basis) or 46.2 lbs/hr (at 10% moisture). In order to meet this constraint, 33% of the cardboard must be fed to the bioprocessing section of the tactical refinery. This will require that cellulase enzymes (that hydrolyze cellulose) be used to carry out hydrolysis in addition to amylases (hydrolyze starch). The fraction of cardboard to be directed to bioprocessing is not optimized for this scenario, since the cardboard contains an organic—lignin—that is not hydrolyzed and will be recycled back to the BioMax 15®. (2) For purposes of V2 model, we have used BioMax 50° efficiency for electrical energy production (at 20.7%). The efficiency of converting the energy content of the organic material to gas is assumed to be 80%. This is lower than the 85% assumed for BioMax 50®. The net energy produced accounts for power consumed by grinder (specified at 5 HP for 1 hour, compared to 2 hours for BioMax 15®), but does not include other power that may be required for pumps associated with filtration of various liquid streams. (3) The results in Table 4 (for the V2 model) are for food waste containing 50% moisture, slop at 90% moisture, and cardboard, plastic and paper waste at 10% moisture. The hydrolysis of starch by enzymes is assumed to occur with 90% yield, and the hydrolysis of cellulose by the cellulase enzymes also at 90% yield. Fermentation of the resulting hexoses is assumed to be achievable at 90% yield. Xylose is assumed not to be fermented for purposes of this calculation, although technology does exist to do this, and can be added later. (4) The fermentation is assumed to require 24 hours to complete with a final concentration of 7% ethanol. After fermentation, a combination of distillation and drying (of the ethanol distillate) occurs with 95% recovery of the product, with the remaining ethanol being found in the aqueous stream from the bottom of the column. Part of this stream will be recycled to the process (temperature is at 100 C), and the rest of the hot water may be suitable for asepticetically processing eating utensils. While “sterilized”, this water contains unfermented sugars, yeast remains, etc. Clean-up (filtration) of the water will be needed.

TABLE 1
Number of troops 800
Conditions

| | | |
|---|---|---|
| Moisture in food | 50% | Xw |
| Moisture in | 10% | |
| Paper/plastic | | |
| Moisture in slop | 90% | |
| Density, ethanol, 20 C | 0.78 | g/mL |
| Density, water 20 C | 1.00 | g/mL |
| L/gal | 3.78 | |

Conversion and Recovery Factors

| | | |
|---|---|---|
| Separation Ethanol | 0.95 | Ys |
| Enzyme Reaction | 0.9 | Ye |
| Distillation Energy | 20,000 | Btu/gal |
| Ethanol Drying Energy | 10,000 | Btu/gal |
| Density 99.6% Ethanol | 6.6 | lbs/gal est |
| Fermentation | 0.900 | Yf |
| Distillation and | 0.950 | Ym |
| drying | | |
| Gasifier to Heat | 0.800 | Yg |
| Gasifier to | 0.207 | Yl |
| Electricity | | |
| Delta H Water | 1000 | btu/lb |
| % Water recycle | 65% | |
| lbs/gal water | 8.34 | |

TABLE 2
Weights of materials generated by 800 troops.
Materials

| | | | Units |
|---|---|---|---|
| Input | | | |
| Kitchen Waste | | 2904.3 | lb |
| Kitchen Oil | | 0.3 | lb |
| Enzyme | | 50.0 | L |
| Yeast | | 10.0 | lb |
| Water | | 2121.9 | lb |
| Output | | | |
| Metals | | 61.6 | lb |
| CO2 | | 222.9 | lb |
| Eth/H2O | 0.39% | 36.7 | gallons |
| Solids | | | lb |
| Ethanol | 99.60% | 33.5 | gallons |

TABLE 3
Composition of
waste for V2 - 800 person case

| | Weight Per | | | | | | | | | | |
|---|---|---|---|---|---|---|---|---|---|---|---|
| | Person Per | Weight Per | | | | | | | | | Gasification |
| | Meal | day for 800 | | | | | | | | | Heat of |
| | (lb/(person * | troops, 3 | Constituents for Fermentation Calculations | | | | | | | | Combustion |
| | meal)) | meals/day, | | | | Hemi- | | | | | (Btu/lb), Dhi |
| Category | [2] | Xi | Starch | Cellulose | Sugar | cellulose | Xylan | Ash | Fat, Protein | Total | [2] |
| Cardboard | 0.183 | 439.20 | | 73.00% | | 17.00% | 7.00% | 2.00% | 1.00% | 100.00% | 7370 |
| Food | 0.317 | 760.80 | 60.00% | | 30.00% | | 3.00% | 2.00% | 5.00% | 100.00% | 2370 |
| Slop Food | 0.251 | 602.40 | 60.00% | | 30.00% | | 3.00% | 2.00% | 5.00% | 100.00% | 1000 |
| Metal-Aluminum | 0.003 | 7.20 | | | | | | | | | 0 |
| Metal-Iron | 0.014 | 33.60 | | | | | | | | | 0 |
| Paper-Brown | 0.198 | 475.20 | 11.00% | 59.00% | | 20.00% | 7.00% | 2.00% | 1.00% | 100.00% | 7370 |
| Paper-Wax | 0.085 | 204.00 | 11.00% | 59.00% | | 20.00% | 7.00% | 2.00% | 1.00% | 100.00% | 9267 |
| Plastic- | 0.001 | 2.40 | | | | | | | | | 9560 |
| Polyethylene | | | | | | | | | | | |
| Terephthalate | | | | | | | | | | | |
| Plastic- | 0.026 | 62.40 | | | | | | | | | 20043 |
| Polyethylene | | | | | | | | | | | |
| Polypropylene | | | | | | | | | | | |
| Plastic- | 0.081 | 194.40 | | | | | | | | | 17111 |
| Polystyrene | | | | | | | | | | | |
| Unopened-MREs | 0.008 | 19.20 | | | | | | | | | 5458 |
| Wood | 0 | 0.00 | 0.00% | 52.00% | | 38.00% | 7.00% | 2.00% | 1.00% | 100.00% | 8189 |
| Opened-MRE | 0.041 | 98.40 | | | | | | | | | 10275 |
| Inner Packaging | | | | | | | | | | | |
| MRE-Heaters | 0.002 | 4.80 | | | | | | | | | 11019 |
| Waste Oil | 1.65E−04 | 0.26 | | | | | | | | | 16809 |

| | | | |
|---|---|---|---|
| Total | 2904.26 | Separated Fats, | 10,000 |
| | | Proteins | |

TABLE 4

| Description | Input | Output |
|---|---|---|
| Kitchen waste, some cardboard to | 1659 | 45.1 |
| bioprocessing, lbs/day | | |
| Cardboard, paper, plastic to BioMax 15, | 1106 | 36.3 |
| lbs/day | | |
| Kitchen oil, protein, fat, lbs/day | 17.6 | 0 |
| Metals, lbs/day | 61.2 | 61.2 |
| Enzyme, L/day | 50 | 50 |
| Yeast, lbs/day | 10 | >10 |
| Added Water, at 65% recycle (lbs per day/ | (173/21) | (173/21) |
| gal per day) | | |
| BioMax Grinder, Kw | 3.8 | 0 |
| | (1 hrs, 5 Hp) | |
| Bioprocess macerator, Kw | 0.37 | 0 |
| | (1 hr, 0.5 HP) | |
| Process Heat Energy, Btu/hr | (propane) | 200,000 |
| Use for distillation | | (42,000) |
| Use for heating water | | (158,000) |
| BioMax “Waste Heat”, Btu/hr | 0 | 51,200 |
| Electrical Energy², Kw | | |
| Average | 0 | 15.1 |
| Minimum | 0 | 11.2 |
| Ethanol lbs/hr | 0 | 9.2 |
| Gal/hr | 0 | 1.4 |

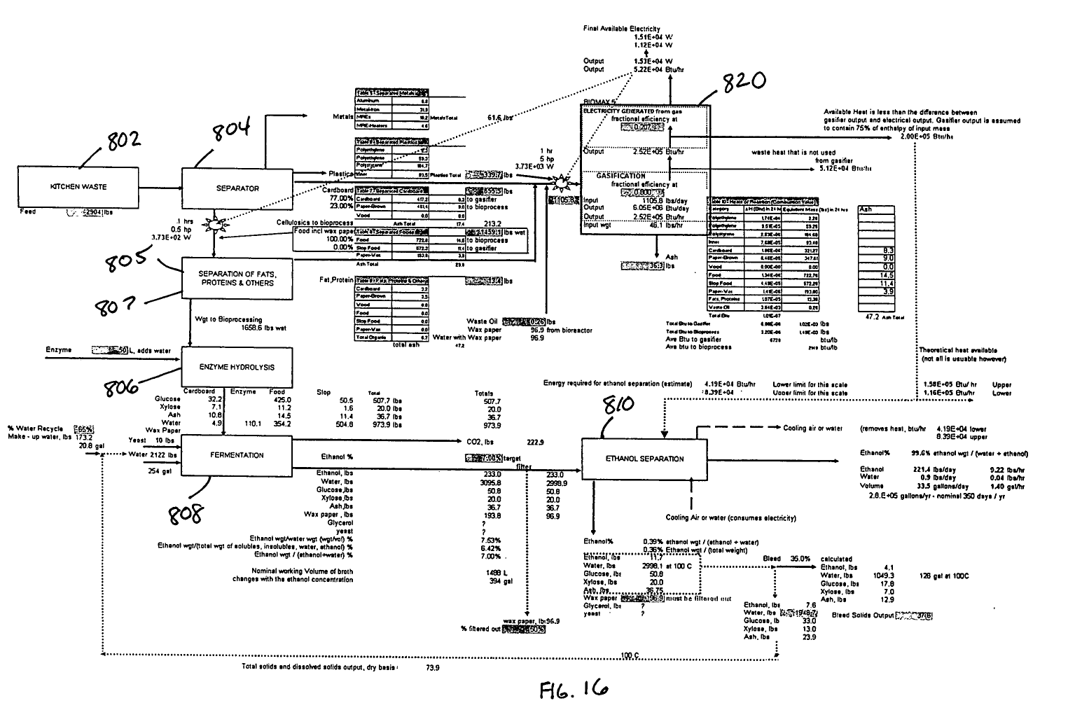

Referring now to FIG. 16, a detailed schematic and mathematical model supporting the data included in the above tables is provided. According to this schematic, kitchen waste 802 (as detailed in Table 3 above) is delivered to a separator 804 that removes metals, plastics and cardboard. The metal is separated out so that it does not enter the gasification unit 820. The other waste materials pass through a 5 HP grinder (such as the BioMax 15® unit) and are ground to a suitable size for introduction into the gasification unit 820. After the waste is ground, they enter the gasifier and a producer gas (low in nitrogen) is made. This gas then goes to the engine/generator set (not shown separately in this diagram) and is used to generate electricity. Some of the electricity is needed to run the rest of the system, while the remaining power available for other sources.

The other wastes that have been separated out by the separator 804 (e.g., uneaten food, fats, oils and paper wastes) is passed through a 0.5 HP grinder 805, which is powered by electricity generated through the biorefinery. At start-up, JP 8 or gasoline can be used to ignite the internal combustion engine that powers the generator set. Once the process begins, the fuel will be cut back and supplanted by the gas stream from the gasification unit 820.

After the additional wastes pass through the second grinder 805, the fats and oils are separated by flotation and they too are skimmed off and sent to the gasification unit 820. Next, the wastes undergo a hydrolysis process 806, in which enzymes are added to break down the starch and other material (e.g., cellulose in card board for example) to glucose. Finally, the glucose is fermented to ethanol in a fermentation step 808.

The fermentation broth that contains ethanol is sent to a distillation column 810 and separated into an ethanol rich phase which is subsequently dried using a molecular sieve. A water rich phase also occurs in which a small amount of ethanol comes out of the bottom of the distillation column 810.

In alternative embodiments according to this exemplary example, an inline system can be constructed wherein wastes are separated and fermented within the same vessel, with the bioreactor and fermentation resulting in the separation (by flotation and settling) of solid materials. These materials are then collected and formed into pellets. The pellets are required by the gasification unit for the purposes of controlling the reaction, and being able to have the right size and density for metering the solids into the gasifier.

### Example 5

### Gasification System—Packaging Waste from 800 Troops (V1)

In this Example, packaging waste (e.g., cardboard and paper) goes directly into a gasifier and the system does not process biological or food waste. The model does not account for externally supplied propane that is needed to start up the BioMax unit. Experimental assumptions are as follows: (1) Ash content is assumed to be low at ˜2%; (2) A conversion efficiency in which 85% of the Btu content of the packaging waste is assumed to be converted to a burnable gas in the gasification step. This was back-calculated from the BioMax 50® specifications, and assumed as an energy content of the wood of 8000 Btu/lb, lower heating value; (3) Of the gas that is formed and fed to the internal combustion engine of the generator set, 207% of the enthalpy of combustion of the producer gas goes into electricity. The remaining enthalpy (heat) is assumed to be a potential source of heat/process energy. Some of this energy will be available at a temperature that can drive heating of water or a distillation/adsorption process; (4) Not accounted for in the energy balance is the propane that will be needed to start-up the gasifier, which could add significant cost to the calculation; (5) The electrical power consumption is for the grinder only, and does not include the electrical control system nor pumps or mixers. The grinder calculation is based on 5 HP and a period of use of 2 hours to grind 3000 lbs of material before it is fed into the gasifier. Since the gasifier will consume 3000 lbs of material per day, there will need to be storage of the ground material since it will only be fed at a rate of 125 lbs per hour; (6) The efficiency of converting the inherent enthalpy (calculated as combustion value) of the cardboard/paper/plastic waste to a gas is assumed to be 85%, while the efficiency of converting the resulting gas to electricity is calculated at 21%.

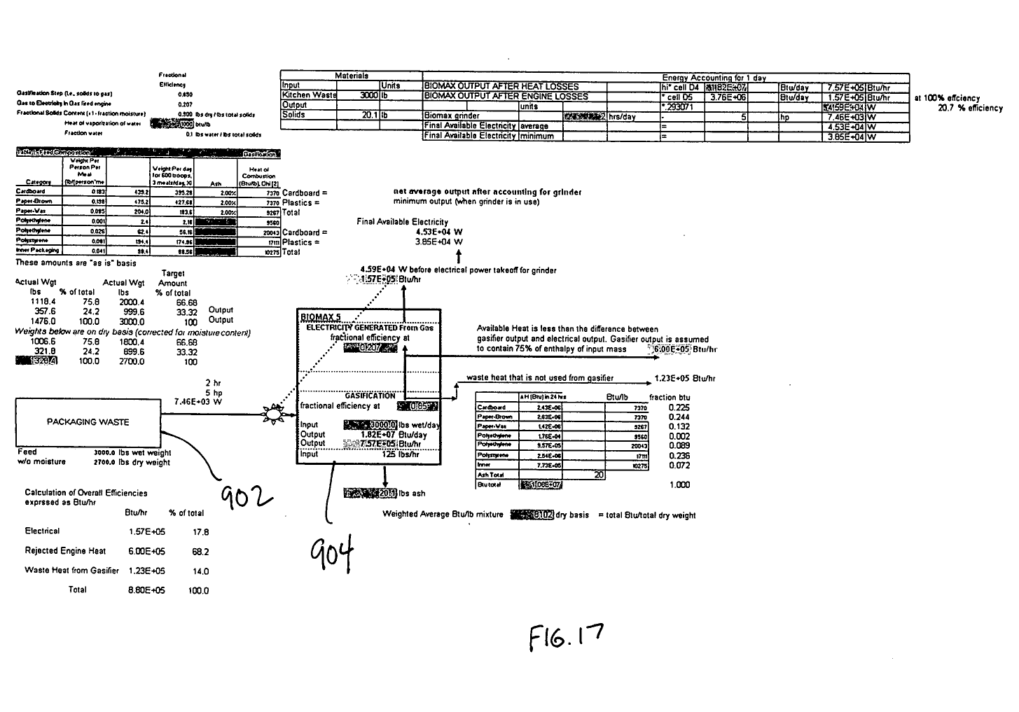

Referring now to FIG. 17, a detailed schematic and mathematical model showing exemplary data in accordance with this model is provided. According to this schematic, packaging waste 902 (as detailed in Table 1 below) is delivered to a gasification unit 904 (such as the BioMax 50® unit) and a producer gas is made. This gas then goes to the engine/generator set (not shown separately in this diagram) and is used to generate electricity.

Calculated results for several cases using this exemplary process are provided in Table 2 below.

TABLE 1
Feed Composition

| | | | | | Gasification |
|---|---|---|---|---|---|
| | Weight Per Person Per | | Weight Per day | | Heat of |
| | Meal (lb/(person * meal)) | | for 600 troops, 3 | | Combustion |
| Category | [2] | | meals/day, Xi | Ash | (Btu/lb), Dhi [2] |
| Cardboard | 0.183 | 439.2 | 395.28 | 2.00% | 7370 |
| Paper-Brown | 0.198 | 475.2 | 427.68 | 2.00% | 7370 |
| Paper-Wax | 0.085 | 204.0 | 183.6 | 2.00% | 9267 |
| Plastic- | 0.001 | 2.4 | 2.16 | | 9560 |
| Polyethylene | | | | | |
| Terephthalate | | | | | |
| Plastic- | 0.026 | 62.4 | 56.16 | | 20043 |
| Polyethylene | | | | | |
| Polypropylene | | | | | |
| Plastic-Polystyrene | 0.081 | 194.4 | 174.96 | | 17111 |
| Opened-MRE | 0.041 | 98.4 | 88.56 | | 10275 |
| Inner Packaging | | | | | |

TABLE 2
Summary of V3 Calculated Inputs/Outputs from MTR © Model

| Description | Input | Output |
|---|---|---|
| Packaging waste - cardboard, paper, | 3000 | 20¹ |
| plastics, lbs/day | (at 2% moisture) | |
| Kitchen Oil, lbs | 0 | 0 |
| Metals, lbs | 46.2 | 46.2 |
| Enzyme, lbs | 0 | 0 |
| Yeast, lbs | 0 | 0 |
| Added Water, lbs | 0 | 0 |
| Power (Grinder), Kw | 7.5 | 0 |
| (2 hrs, 5 Hp) | | |
| Process Heat Energy, Btu/hr | (propane) | 600,000 |
| BioMax “Waste Heat”, Btu/hr | 0 | 123,000 |
| Electrical Energy², Kw | | |
| Average | 0 | 45.3 |
| Minimum | 0 | 38.5 |
| Ethanol lbs | 0 | 0 |
| gal | 0 | 0 |

¹Ash is assumed to pass through the system unconverted
²These values are lower than the gross output of BioMax 50 since there is power consumed by a 5 HP grinder

### Example 6

### Hybrid Biorefinery Design

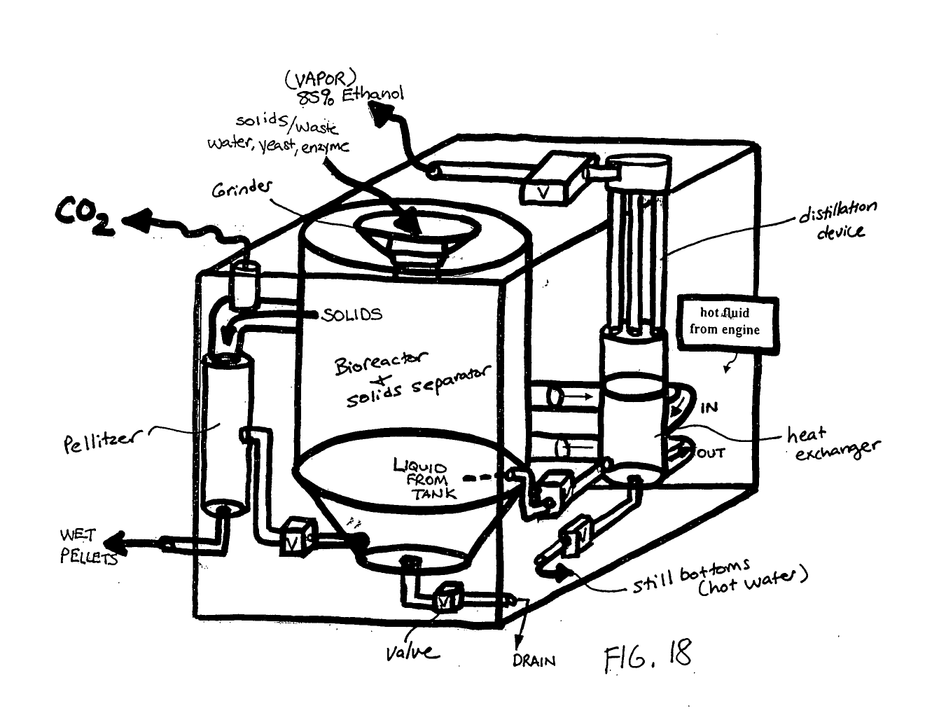

According to this example, a biorefinery apparatus is designed and consists of a conical tank in which a mixture of waste materials are fermented using yeast. The fermentation process results in the separation of the solid non-fermentable waste materials from the yeast itself, as well as the conversion of starch and sugars into ethanol. The solid material floats, and is skimmed off and compacted into a pellet form. The yeast settles to the bottom of the conical tank and is collected. The mixture of the two materials are then combined and pelletized to form chip-sized materials that are fed into the gasifier. The liquid solution contains ethanol, derived from the fermentation of the starch and sugars in the waste material by yeast. This liquid is then distilled to give an 85% ethanol overhead product, and a bottoms product (water product from the distillation column). The 85% product is with output from the gasifier to run the engine/generator set. Concepts of the current design and the method in which these would be integrated are shown in FIG. 18, where the large tank is the fast fermenter, the vessel to the left is the pellitizer, and the column to the right of the fermenter is the distiller.

The central part of the tactical biorefinery is the vessel in which bioreaction (fermentation) and simultaneous solid separation occurs. The type of yeast that is used does not require precooking of the solid material, and therefore greatly simplifies the design. As the fermentation occurs the solid material which initially resembles a wet, solid mass separates into its components. As the fermentation is completed, the yeast cells settle to the bottom of the tank, with their settling being accelerated by the sloped sides of the cone (that provides incline) at the bottom of the tank.

The run was carried out using material provided through Defense Life Sciences. This material simulates MRE/kitchen and dining waste. The tank used for testing was a conical 50 gallon tank. While the tank was 50 gallons in volume, only 9.5 gallons of feed mixture was available.

The preparation and loading steps of this experiment were conducted manually; however, it should be appreciated that in other bioprocessing modules, a shredder may be mounted onto the tank where solid wastes, water, enzyme, and yeast can be fed. Once the material enters the tank, the fermentation process begins. The manner in which the bioreactor was loaded involved multiple steps. These steps are included below. A 100 mL sample was taken at the beginning of the run for purposes of measuring initial sugar and ethanol content. The amount of starch added was calculated as a ratio of sugar that was found in this sample, and which prior work had shown to be constant at 0.76 starch:sugar. The combined glucan from both sugar and starch is used to calculate the ethanol yield.

The fermentation resulted in formation of gas (CO₂) which was vented through an exhaust duct. The tank was allowed to sit for 24 hours, without agitation. The next day there were no visible bubbles and no strong ethanol smell. The cells apparently settled to the bottom of the tank, while solids consisting of plastic/paper/cardboard pieces floated to the top and actually protruded above the liquid. When the bottom valve was opened, a thick white liquid followed by a brown sludge drained from the cone. The volume collected filled 1.5 buckets. The liquid above the cone showed a gradient of color, and became progressively lighter with fluid height.

Tank Loading Steps: 1) Water (minus water fraction from wet MRE mixture and water from yeast mixture); 2) MRE Food (Used dried MRB=1180 g, Wet M mix (63% dry)=1275.6 g); 3) Yeast (500 g block “Safale S-04 Dry Ale Yeast”, Fermentis, France), prepared beforehand by hydrating 30 min untouched and 30 min gently stirred in 5.5 L water; 4) Enzyme; 5) Dry Solids; 6) MRE Inner packaging (cut 1.5×1.5 in), prepared beforehand; 6) Tank contents stirred with dowel for 3 minutes and 7) Sample taken.

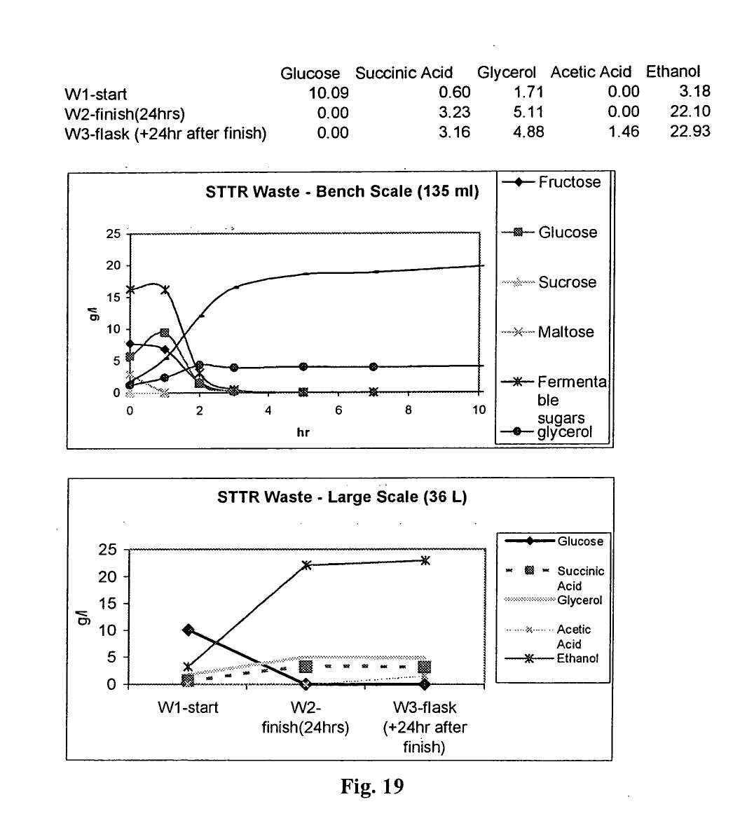

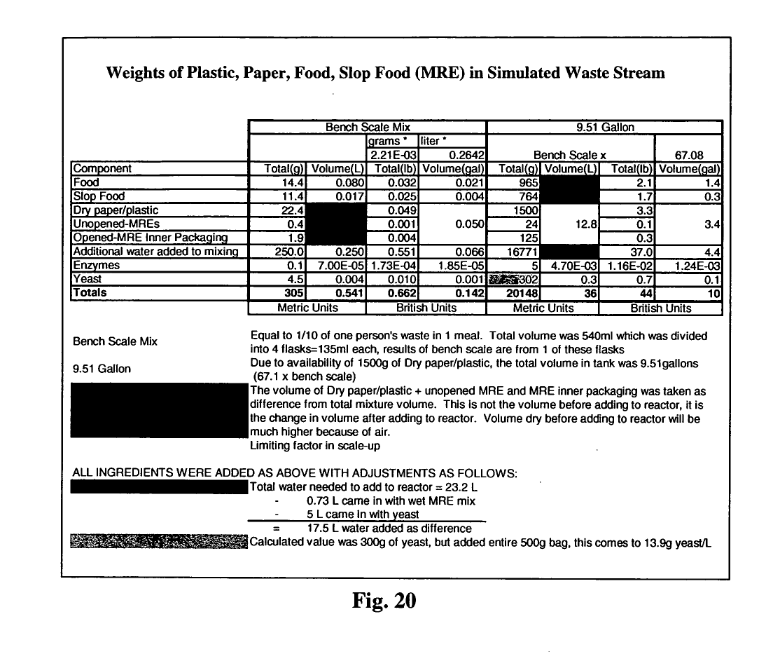

A concurrent shake-flask fermentation showed that most of the fermentable substrate (starch and sugars) had been converted to ethanol in 4 hours. Analysis also showed that the starting slurry contained about 3 g/L or 0.3% ethanol that was probably introduced with the yeast (sample W-1 in FIG. 19). After 24 hours the ethanol content was 22 g/L (W-2) which was comparable to the ethanol content in the flask which was achieved in approximately 4 hours. Sample W-3 shows no further change in the ethanol content after a total of 48 hours. FIG. 19 also shows the fermentation time course for the bench-scale (135 mL) and pilot scale (36 L) runs. The bench-scale run corresponds to about 90% theoretical yield. FIG. 20 shows the weights of different constituents used to make up the simulated waste stream.

After the run was completed, the solids were kept for 40 hours at refrigerated temperature. The liquid was siphoned off, and 1800 g of wet sludge was obtained after either filtration or centrifugation (½ was filtered, ½ was centrifuged (10 min) to produce 1788 g sludge). While centrifugation will not be carried out in a tactical biorefinery, it was used here to more rapidly prepare a material suitable for forming pellets.

Various approaches were attempted to obtain solid pellets that would be suitable for the gasifier, from the solid (sludge) material. These included making cookies, pushing the material through a cold pipe or sausage forming device. Success was finally obtained by pushing the material through a heated pipe. This caused the plastic to melt and serve as a binder to hold the other material together. One tray of heated pipe was put in small oven at 71° C., the logs dried hard and not crumbly. The hard logs should be able to break, but should not crumble when totally dry.

The appended claims set forth some aspects of the integrated thermochemical and biocatalytic energy production system described in its various embodiments believed to be patentable to Applicants. However, the various embodiments disclosed herein may include other inventions patentable to Applicants. Moreover, while exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An apparatus for the processing organic waste into an energy source, comprising:
a fermenter adapted to receive organic waste;
a distillation column in fluid communication with the fermenter and adapted to recover alcohol produced by the fermenter; and
a gasifier adapted to receive solid portions of the organic waste, the gasifier being configured to produce a producer gas adapted to be used for generating electricity.

2. The apparatus of claim 1, wherein the producer gas comprises a hydrocarbon fuel.

3. The apparatus of claim 2, further comprising an electrical generator adapted to receive the hydrocarbon fuel to generate the electricity.

4. The apparatus of claim 1, further comprising a grinder, the grinder being operatively associated with the fermenter to grind the organic waste.

5. The apparatus of claim 1, further comprising a separator in communication with the fermenter, the separator being adapted to receive the organic waste and separate it into high and low moisture content organic wastes.

6. The apparatus of claim 1, further comprising a dryer operatively associated with a means for communicating the solid portions of the organic waste to the gasifier.

7. The apparatus of claim 6, further comprising a means for conveying waste heat from the gasifier to the dryer.

8. The apparatus of claim 4, further comprising a second grinder for processing a portion of the organic waste.

9. The apparatus of claim 1, further comprising a heat exchanger for receiving waste heat from the gasifier.

10. The apparatus of claim 1, further comprising a hydrolysis reaction chamber, the hydrolysis reaction chamber being configured to receive a portion of the organic waste and provide hydrolyzed organic waste to the fermenter.

11. A method of treating an organic waste comprising:
separating an organic waste stream into high and low moisture content organic waste streams;
subjecting the low moisture content organic waste stream to a gasification process to generate a producer gas; and
subjecting the high moisture content organic waste to a fermentation process to produce a mixture of ethanol and water.

12. The method of claim 11, further comprising conveying waste heat from the gasification process to a distillation column.

13. The method of claim 12, further comprising separating the mixture of ethanol and water by the distillation column.

14. The method of claim 11, further comprising subjecting the high moisture content organic waste to a grinder, the grinder being adapted to separate fats, proteins and oils from the waste.

15. The method of claim 14, wherein the separated fats, proteins and oils are delivered to the gasification process to generate producer gas.

16. The method of claim 11, further comprising subjecting the high moisture content organic waste to an enzyme hydrolysis process, the enzyme hydrolysis process producing glucose.

17. The method of claim 16, wherein the step of subjecting the high moisture content organic waste to the fermentation process to produce a mixture of ethanol and water comprises fermenting the glucose that is produced by the enzyme hydrolysis process.

18. The method of claim 12, further comprising recovering vapors from the distillation column and mixing the vapors with the producer gas to produce a fuel for use as an energy source.

19. A method of treating an organic waste comprising:
subjecting the organic waste stream to a fermentation process to produce an ethanol and water mixture;
separating any residual solids from the fermentation step from the ethanol and water mixture; and
subjecting the any residual solids to a gasification process to generate a producer gas.

20. The method of claim 19, further comprising separating the ethanol and water mixture by a distillation process.

21. The method of claim 20, further comprising capturing waste heat from the gasification step and providing it to the distillation process.

22. The method of claim 20, further comprising recovering vapors from the distillation process and mixing the vapors with the producer gas to produce a fuel for use as an energy source.
